(19) United States
(12) Patent Application Publication
(10) Pub. No.: US 2025/0176860 A1
(43) Pub. Date: Jun. 5, 2025

(54) FINGERPRINT IMAGING DEVICE

(71) Applicant: NEC Corporation, Minato-ku, Tokyo (JP)

(72) Inventor: Yoshinori KODA, Tokyo (JP)

(73) Assignee: NEC Corporation, Minato-ku, Tokyo (JP)

(21) Appl. No.: 18/843,736

(22) PCT Filed: Mar. 28, 2022
(86) PCT No.: PCT/JP2022/014820

§ 371 Date: Sep. 4, 2024

(51) Int. Cl. A61B 5/1172; G06V 10/141; G06V 40/13

(52) U.S. Cl. CPC A61B 5/1172; G06V 10/141; G06V 40/13; A61B 2503/045

(57) ABSTRACT

An object of the present disclosure is to provide a fingerprint imaging device capable of imaging a fingerprint by switching a plurality of imaging devices. A fingerprint imaging device includes a housing that has an upper portion in which an opening is formed; a strut that is attached to the housing to be perpendicular to the upper portion of the housing; a rotary table that is rotatably attached to the strut with the strut as a rotation axis; a first imaging device that is disposed on an upper surface of the rotary table; and a second imaging device that is disposed in the housing to be immediately below the opening and below the rotary table. A part of the rotary table is exposed from the housing. The first imaging device is moved to a position immediately below the opening as the rotary table is rotated.

### TECHNICAL FIELD

The present disclosure relates to a fingerprint imaging device that images a fingerprint.

### BACKGROUND ART

In the related art, various apparatuses for imaging a fingerprint have been proposed. As an example of such an apparatus, a personal identification apparatus disclosed in Patent Literature 1 includes a plurality of light sources that irradiate a fingerprint of a finger with transmitted light having different irradiation angles, a finger support portion that supports a fingerprint surface of the finger in a non-contact state, and an imaging unit that images light transmitted through the fingerprint of the finger supported by the finger support portion to obtain a plurality of fingerprint images corresponding to the fingerprint.

### CITATION LIST

### Patent Literature

Patent Literature 1: Japanese Unexamined Patent Application Publication No. 2007-207097

### SUMMARY OF INVENTION

### Technical Problem

However, since the personal identification apparatus disclosed in Patent Literature 1 images a fingerprint using a single imaging unit, there is a problem that it is not possible to image a fingerprint by switching a plurality of imaging devices.

An object of the present disclosure is to provide a fingerprint imaging device capable of imaging a fingerprint by switching a plurality of imaging devices in view of the above-described problems.

### Solution to Problem

A fingerprint imaging device according to an exemplary aspect includes
a housing that has an upper portion in which an opening is formed:
a strut that is attached to the housing to be perpendicular to the upper portion of the housing:
a rotary table that is rotatably attached to the strut with the strut as a rotation axis:
a first imaging device that is disposed on an upper surface of the rotary table: and
a second imaging device that is disposed in the housing to be immediately below the opening and below the rotary table,
in which a part of the rotary table is exposed from the housing, and
the first imaging device is moved to a position immediately below the opening as the rotary table is rotated.

A fingerprint imaging device according to another exemplary aspect includes
a housing that has an upper surface in which an opening is formed:
a strut that is attached to the housing to be perpendicular to the upper surface of the housing:
a rotary table that is rotatably attached to the strut with the strut as a rotation axis: and
a plurality of first imaging devices disposed at different positions on the upper surface of the rotary table:
in which a part of the rotary table is exposed from the housing, and
each of the plurality of first imaging devices is moved to a position immediately below the opening as the rotary table is rotated.

### BRIEF DESCRIPTION OF DRAWINGS

FIG. 1 is a perspective view illustrating a fingerprint imaging device according to a first example embodiment.

FIG. 2 is a sectional view taken along a sectional line I-I illustrated in FIG. 1.

FIG. 3 is a view illustrating an upper surface of a rotary table according to the first example embodiment.

FIG. 4 is a sectional view illustrating main components of the fingerprint imaging device according to the first example embodiment.

FIG. 5 is an elevation view illustrating a rotation mechanism according to a second example embodiment.

FIG. 6 is an elevation view illustrating the rotation mechanism according to the second example embodiment.

FIG. 7 is a view illustrating a lower surface of a guide according to the second example embodiment.

FIG. 8 is a sectional view of a fingerprint imaging device according to the second example embodiment as viewed from a transverse direction.

FIG. 9 is a view illustrating an example of a rotary table according to a third example embodiment.

FIG. 10 is a block diagram illustrating a configuration of a motor control apparatus according to the third example embodiment.

FIG. 11 is a flowchart illustrating an example of processing executed by the motor control apparatus according to the third example embodiment.

FIG. 12 is a view illustrating an example of a rotation mechanism according to a fourth example embodiment.

FIG. 13 is an elevation view of a fingerprint imaging device according to a sixth example embodiment as viewed from a transverse direction.

FIG. 14 is an elevation view of the fingerprint imaging device according to the sixth example embodiment as viewed from a longitudinal direction.

FIG. 15 is a diagram illustrating a configuration of a notification control apparatus according to a seventh example embodiment.

FIG. 16 is a flowchart illustrating an example of processing executed by the notification control apparatus according to the seventh example embodiment.

FIG. 17 is a diagram illustrating a configuration of a notification control apparatus according to an eighth example embodiment.

FIG. 18 is a flowchart illustrating an example of processing executed by the notification control apparatus according to the eighth example embodiment.

FIG. 19 is a diagram illustrating a configuration of a light source control apparatus according to a ninth example embodiment.

FIG. 20 is a flowchart illustrating an example of processing executed by the light source control apparatus according to the ninth example embodiment.

FIG. 21 is a flowchart illustrating an example of processing executed by a light source control apparatus according to a tenth example embodiment.

FIG. 22 is a view illustrating an example of a rotary table according to an eleventh example embodiment.

FIG. 23 is a sectional view illustrating main components of a fingerprint imaging device according to a twelfth example embodiment.

### EXAMPLE EMBODIMENT

### First Example Embodiment

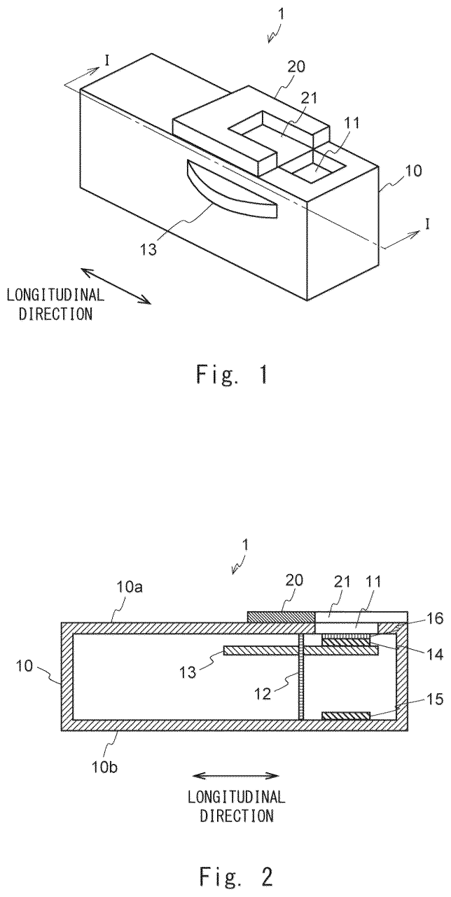

Hereinafter, exemplary example embodiments will be described with reference to the drawings. FIG. 1 is a perspective view illustrating a fingerprint imaging device 1 according to a first example embodiment.

The fingerprint imaging device 1 includes a housing 10, and a guide 20 disposed on an upper surface of the housing 10. An opening 11 is formed in an upper portion 10a of the housing 10. A finger as an imaging target is placed on the opening 11. The opening 11 preferably has a size capable of imaging the entire fingerprint of one finger.

The guide 20 is used in a case where a target person of which the fingerprint is to be imaged is a newborn baby. The guide 20 is disposed on the upper surface of the housing 10 to slide in the longitudinal direction of the fingerprint imaging device 1. The guide 20 has a cutout portion 21 formed over the upper surface and the lower surface of the guide 20. The cutout portion 21 is formed such that the size when viewed from the upper surface side of the guide 20 is equal to or greater than the size of the opening 11. In a state where the guide 20 is moved to a position where the opening 11 and the cutout portion 21 are continuous, a finger of a newborn baby who is a target person is placed on the cutout portion 21. In other example embodiments, the fingerprint imaging device 1 may not include the guide 20.

FIG. 2 is a sectional view taken along a sectional line I-I illustrated in FIG. 1. The fingerprint imaging device 1 includes a strut 12, a rotary table 13, a first imaging device 14, a second imaging device 15, and a protective plate 16.

The strut 12 is attached to the housing 10 to be perpendicular to the upper portion 10a and a lower portion 10b of the housing 10. The rotary table 13 is rotatably attached to the strut 12 with the strut 12 as a rotation axis. The rotary table 13 is disposed on the upper portion side of the housing 10.

The first imaging device 14 is a device that images a finger as the imaging target to generate a fingerprint image. When a button (not illustrated) for instructing the imaging is pressed, the first imaging device 14 can perform imaging. The first imaging device 14 is disposed on the upper surface of the rotary table 13. The first imaging device 14 is moved to a position immediately below the opening 11 as the rotary table 13 is rotated.

The protective plate 16 is a component for protecting the first imaging device 14, and is disposed on the upper surface of the first imaging device 14. The protective plate 16 is made of a transparent material such as glass or plastic. Note that, in other example embodiments, the protective plate 16 may be omitted.

The second imaging device 15 is a device that images a finger as the imaging target to generate a fingerprint image. When a button (not illustrated) for instructing the imaging is pressed, the second imaging device 15 can perform imaging. The second imaging device 15 is disposed immediately below the opening 11 and below the rotary table 13 in the housing 10.

In the present example embodiment, the first imaging device 14 can be an imaging device for imaging a fingerprint of a newborn baby. The second imaging device 15 can be an imaging device for imaging a fingerprint of a target person other than a newborn baby, for example, an adult. In this case, a resolution of the first imaging device 14 is preferably higher than that of the second imaging device 15.

In other example embodiments, both the first imaging device 14 and the second imaging device 15 may be imaging devices for imaging a fingerprint of a target person other than a newborn baby. In this case, the resolution of the first imaging device 14 and the resolution of the second imaging device 15 can be the same.

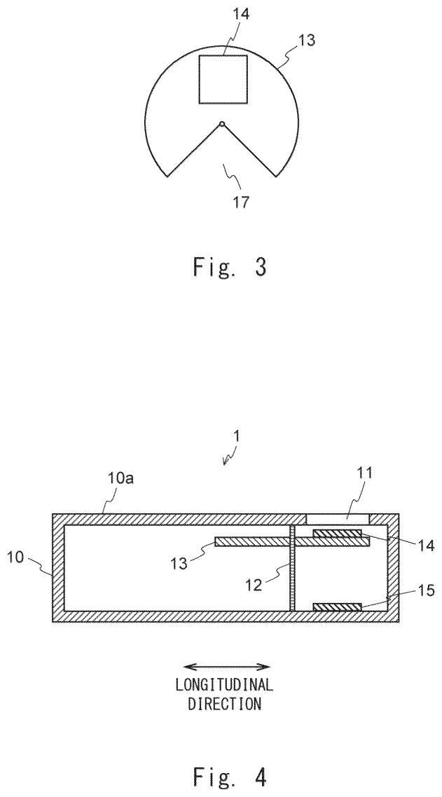

FIG. 3 is a view illustrating an upper surface of the rotary table 13. As illustrated in FIG. 3, the first imaging device 14 is disposed on the rotary table 13. When the rotary table 13 is rotated so that the first imaging device 14 is moved to a position immediately below the opening 11, fingerprint imaging by the first imaging device 14 becomes possible.

A cutout portion 17 is formed in a part of the rotary table 13. When the rotary table 13 is rotated so that the cutout portion 17 is moved to a position immediately below the opening 11, fingerprint imaging by the second imaging device 15 becomes possible.

The rotary table 13 can be made of an opaque material, a translucent material, or a transparent material. In an example embodiment in which the rotary table 13 is formed using an opaque material or a translucent material, in a case where the cutout portion 17 is positioned immediately below the opening 11, fingerprint imaging by the second imaging device 15 becomes possible. On the other hand, in an example embodiment in which the rotary table 13 is formed using a transparent material, in a case where the first imaging device 14 is not positioned immediately below the opening 11, fingerprint imaging by the second imaging device 15 becomes possible. In an example embodiment in which the rotary table 13 is formed using a transparent material, the rotary table 13 may not be provided with the cutout portion 17.

A part of the rotary table 13 is exposed from the housing 10 as illustrated in FIG. 1. A user of the fingerprint imaging device 1 can switch between the first imaging device 14 and the second imaging device 15 to be used for imaging a fingerprint, by rotating the rotary table 13 using the exposed portion of the rotary table 13.

FIG. 4 is a sectional view illustrating main components of the fingerprint imaging device 1 according to the first example embodiment. The fingerprint imaging device 1 includes the housing 10, the strut 12, the rotary table 13, the first imaging device 14, and the second imaging device 15. The housing 10 has the upper portion 10a in which the opening 11 is formed. The strut 12 is attached to the housing 10 to be perpendicular to the upper portion 10a the housing 10. The rotary table 13 is rotatably attached to the strut 12 with the strut 12 as a rotation axis. A part of the rotary table 13 is exposed from the housing 10. The first imaging device 14 is disposed on the upper surface of the rotary table 13, and is moved to a position immediately below the opening 11 as the rotary table 13 is rotated. The second imaging device 15 is disposed immediately below the opening 11 and below the rotary table 13 in the housing 10.

In the fingerprint imaging device 1, by adopting the above configuration, the user can rotate the rotary table 13 exposed from the housing 10, and can switch between the first imaging device 14 and the second imaging device 15 to be used for imaging a fingerprint.

In addition, the first imaging device 14 can be an imaging device for imaging a fingerprint of a newborn baby. In this case, the resolution of the first imaging device 14 can be set to be higher than that of the second imaging device 15. Thus, the fingerprint imaging device 1 can use the first imaging device 14 to image a fingerprint of a newborn baby for which an image with a high resolution is required. In addition, the fingerprint imaging device 1 can use the second imaging device 15 to image a fingerprint of a target person other than a newborn baby for which an image with a relatively low resolution is allowed.

Furthermore, the first imaging device 14 and the second imaging device 15 can be imaging devices for imaging a fingerprint of a target person other than a newborn baby. In this case, the resolution of the first imaging device 14 and the resolution of the second imaging device 15 can be the same. Thus, the fingerprint imaging device 1 can use both the first imaging device 14 and the second imaging device 15 to image a fingerprint of a target person other than a newborn baby.

Furthermore, as illustrated in FIG. 1, the fingerprint imaging device 1 can include the guide 20 disposed on the upper surface of the housing 10 such that the guide 20 slides in the longitudinal direction of the fingerprint imaging device 1. The guide 20 has the cutout portion 21 formed over the upper surface and the lower surface of the guide 20. The cutout portion 21 is formed such that the size when viewed from the upper surface side of the guide 20 is equal to or greater than the size of the opening 11.

Thus, in a state where the guide 20 is moved to a position where the opening 11 and the cutout portion 21 are continuous, a finger of a target person placed on the cutout portion 21 can be separated from the upper surface of the housing 10 by the thickness of the guide 20. Therefore, even when an adult presses a finger of a newborn baby against the periphery of the cutout portion 21 of the guide 20 in a case of imaging the finger of the newborn baby, the finger of the newborn baby can be prevented from excessively approaching the first imaging device 14. As a result, damage to the first imaging device 14 and the protective plate 16 can be prevented. In addition, it is possible to obtain a captured image with a high quality without defocusing or the like.

### Second Example Embodiment

In a second example embodiment, the fingerprint imaging device 1 switches between the first imaging device 14 and the second imaging device 15 to be used for imaging a fingerprint, by user operating the guide 20. Hereinafter, differences from the first example embodiment will be mainly described.

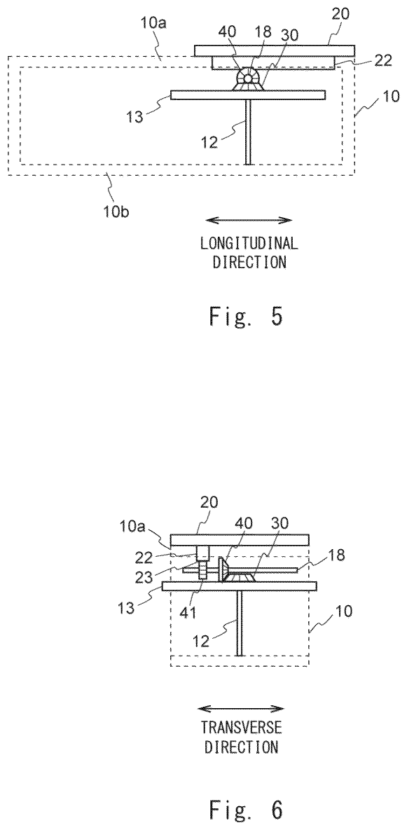

FIGS. 5 to 7 are views illustrating a rotation mechanism for rotating the rotary table 13 according to the second example embodiment. The rotary table 13 can be configured to be rotated in conjunction with the sliding of the guide 20. Note that the rotary table 13 includes the first imaging device 14 and the protective plate 16 (not illustrated) as in the first example embodiment. The configuration of the rotation mechanism using gears illustrated in FIGS. 5 to 7 is an example, and various other types of gears may be adopted. In addition, the number of gears to be used is not limited to the configuration illustrated in FIGS. 5 to 7.

FIG. 5 is an elevation view illustrating the rotation mechanism viewed from the transverse direction of the fingerprint imaging device 1. FIG. 6 is an elevation view illustrating the rotation mechanism viewed from the longitudinal direction of the fingerprint imaging device 1. As illustrated in FIGS. 5 and 6, the fingerprint imaging device 1 further includes a bevel gear 30, a strut 18, a bevel gear 40, and a spur gear 41. The bevel gear 30 is fixed to the upper surface of the rotary table 13, and is rotated about the strut 12 together with the rotary table 13. Note that, in the present example embodiment, the strut 12 extends from the lower portion 10b of the housing 10 to the upper surface of the bevel gear 30.

The strut 18 is rotatably attached to the housing 10 so as to be perpendicular to the strut 12. The bevel gear 40 is fixed to the strut 18 and is rotated about the strut 18. The bevel gear 30 and the bevel gear 40 are arranged to engage with each other.

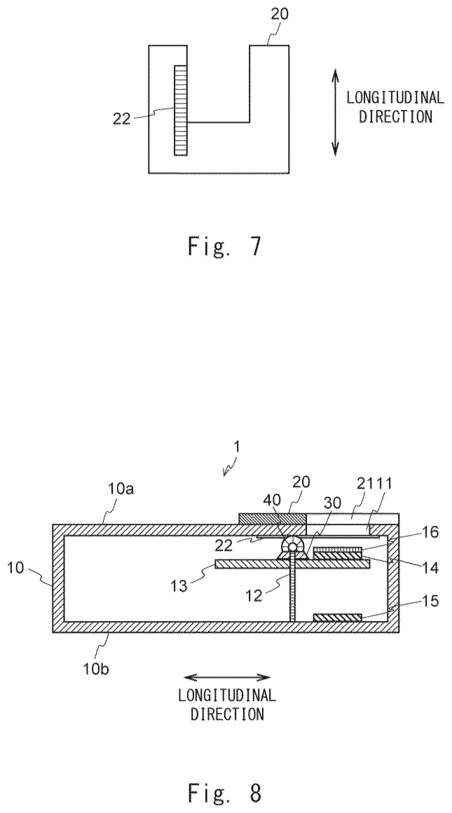

As illustrated in FIG. 6, the gear 41 is further fixed to the strut 18. The gear 41 engages with a plurality of protrusions 23 formed on the lower surface of a projection portion 22 of the guide 20, which projects downward of the guide 20. An opening into which the projection portion 22 of the guide 20 is inserted is formed in the upper portion 10a of the housing 10. FIG. 7 is a view illustrating the lower surface of the guide 20. As illustrated in FIG. 7, the plurality of protrusions 23 are continuously formed on the lower surface of the guide 20 in the longitudinal direction of the fingerprint imaging device 1.

As illustrated in FIG. 8, the size and pitch of the protrusions 23 of the projection portion 22 of the guide 20, the size of the gears 30, 40, and 41, and the size and pitch of the teeth of the gears 30, 40, and 41 can be configured such that the first imaging device 14 is positioned immediately below the opening 11 in a case where the guide 20 is moved to a position where the cutout portion 21 of the guide 20 and the opening 11 are continuous.

On the other hand, the size and pitch of the protrusions 23 of the projection portion 22 of the guide 20, the size of the gears 30, 40, and 41, and the size and pitch of the teeth of the gears 30, 40, and 41 can be configured such that the fingerprint imaging by the second imaging device 15 becomes possible in a case where the guide 20 deviates from the position where the cutout portion 21 of the guide 20 and the opening 11 are continuous.

Specifically, in a case where the rotary table 13 formed of a transparent material is adopted, the size and pitch of the protrusions 23 of the projection portion 22 of the guide 20, the size of the gears 30, 40, and 41, and the size and pitch of the teeth of the gears 30, 40, and 41 are configured such that the first imaging device 14 deviates from the position immediately below the opening 11. Thus, the fingerprint imaging of the imaging target by the second imaging device 15 becomes possible.

In a case where the rotary table 13 formed of an opaque material or a translucent material is adopted, the size and pitch of the protrusions 23 of the projection portion 22 of the guide 20, the size of the gears 30, 40, and 41, and the size and pitch of the teeth of the gears 30, 40, and 41 are configured such that the first imaging device 14 deviates from the position immediately below the opening 11 and the cutout portion 17 of the rotary table 13 is moved to a position immediately below the opening 11. Thus, the fingerprint imaging of the imaging target by the second imaging device 15 becomes possible.

As the guide 20 slides in the longitudinal direction of the fingerprint imaging device 1, the protrusions 23 of the guide 20 rotate the gear 41 engaging with the protrusions 23. When the gear 41 is rotated, the strut 18 to which the gear 41 is fixed is rotated, and the bevel gear 40 fixed to the strut 18 is rotated. When the bevel gear 40 is rotated, the bevel gear 30 engaging with the bevel gear 40 is rotated, and the rotary table 13 to which the bevel gear 30 is fixed is rotated. In this manner, the rotary table 13 can be rotated in conjunction with the sliding of the guide 20.

As described above, in the second example embodiment, the rotary table 13 can be configured to be rotated in conjunction with the sliding of the guide 20. The guide 20 rotates the rotary table 13 such that the first imaging device 14 is moved to a position immediately below the opening 11 in a case where the guide 20 is moved to a position where the cutout portion 21 of the guide 20 and the opening 11 are continuous. In addition, when the guide 20 at a position where the cutout portion 21 of the guide 20 and the opening 11 are continuous is moved away from the opening 11, the guide 20 rotates the rotary table 13 such that the fingerprint imaging of the imaging target by the second imaging device 15 becomes possible. Thus, the user of the fingerprint imaging device 1 can switch between the first imaging device 14 and the second imaging device 15 to be used for imaging a fingerprint, by moving the guide 20.

### Third Example Embodiment

In a third example embodiment, the fingerprint imaging device 1 switches between the first imaging device 14 and the second imaging device 15 to be used for imaging a fingerprint, according to the size of the finger as the imaging target. Hereinafter, differences from the first example embodiment will be mainly described.

The fingerprint imaging device 1 further includes a detection device 50 that detects the size of the finger as the imaging target placed on the opening 11, a motor that rotates the rotary table 13, and a motor control apparatus 70 that controls the rotation of the motor. The detection device 50 and the motor control apparatus 70 are connected via a communication line. The motor control apparatus 70 and the motor are connected via a communication line.

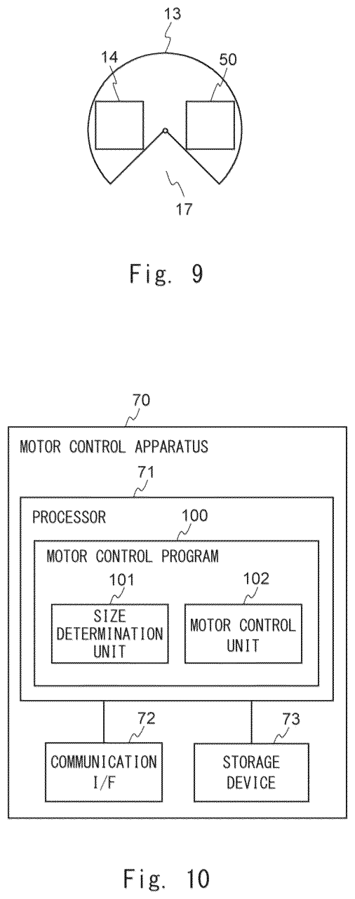

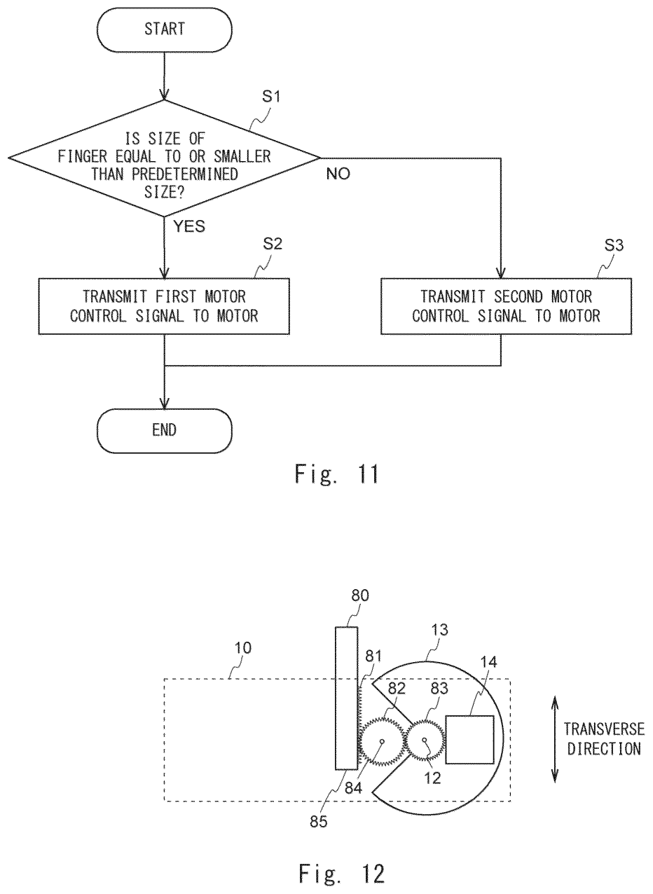

FIG. 9 is a view illustrating the upper surface of the rotary table 13 according to the third example embodiment. As illustrated in FIG. 9, the detection device 50 can be disposed on the upper surface of the rotary table 13. The detection device 50 provides detected information (hereinafter, referred to as “detection information”.) to the motor control apparatus 70 via a communication line. Specific examples of the detection device 50 include an imaging device, a touch sensor, and the like. In a case where an imaging device is adopted as the detection device 50, the detection device 50 provides a captured image to the motor control apparatus 70 as the detection information. In a case where a touch sensor is adopted as the detection device 50, the detection device 50 provides information indicating a region touched by a finger, to the motor control apparatus 70 as the detection information.

The motor is an apparatus that rotates the rotary table 13 under the control of the motor control apparatus 70. In the present example embodiment, the rotary table 13 can be installed on the rotation axis of the motor. In other words, the rotation axis of the motor functions as the strut 12. The motor is attached to the housing 10 such that the rotation axis of the motor is perpendicular to the upper portion 10a of the housing 10.

FIG. 10 is a block diagram illustrating the configuration of the motor control apparatus 70 according to the third example embodiment. Specific examples of the motor control apparatus 70 include a microcomputer and the like. The motor control apparatus 70 includes a processor 71 capable of executing various programs, a communication interface (I/F) 72, and a storage device 73. Specific examples of the processor 71 include various processors such as a central processing unit (CPU) and a micro processing unit (MPU).

The motor control apparatus 70 receives the detection information of the detection device 50 via the communication interface 72. In addition, the motor control apparatus 70 transmits a control signal for controlling the motor to the motor via the communication interface 72.

The storage device 73 stores a motor control program 100 executed by the processor 71 and various kinds of information. The processor 71 executes a method according to the exemplary example embodiment by reading the motor control program 100 from the storage device 73 and executing the motor control program 100. The motor control program 100 includes a size determination unit 101 and a motor control unit 102. Note that the functions of the motor control program 100 may be implemented by an integrated circuit such as a field-programmable gate array (FPGA) or an application specific integrated circuit (ASIC). An integrated circuit such as a processor, an FPGA, and an ASIC corresponds to a computer.

The size determination unit 101 is a program that determines the size of a finger as the imaging target placed on the opening 11 by using the detection information of the detection device 50. Specifically, the size determination unit 101 determines whether or not the size of a finger as the imaging target is equal to or smaller than a predetermined size. The predetermined size can be a typical finger size of a newborn baby.

In a case where an imaging device is adopted as the detection device 50, the size determination unit 101 determines whether or not the size of the finger included in the captured image generated by the detection device 50 is equal to or smaller than the predetermined size. In a case where a touch sensor is adopted as the detection device 50, the size determination unit 101 determines whether or not the size of the region touched by the finger is equal to or smaller than the predetermined size on the basis of the information indicating the region output by the detection device 50.

The motor control unit 102 is a program that controls the rotation of the motor according to a determination result of the size determination unit 101. In a case where it is determined that the size of the finger as the imaging target is equal to or smaller than the predetermined size, the motor control unit 102 transmits a first motor control signal to the motor. The first motor control signal is a control signal for rotating the rotary table 13 such that the first imaging device 14 is moved to a position immediately below the opening 11. The first motor control signal includes information for instructing a rotation angle by which the rotary table 13 should be rotated. The rotation angle can take positive and negative values.

On the other hand, in a case where it is determined that the size of the finger as the imaging target exceeds the predetermined size, the motor control unit 102 transmits a second motor control signal to the motor. The second motor control signal is a control signal for rotating the rotary table 13 such that the fingerprint imaging by the second imaging device 15 becomes possible. The second motor control signal includes information for instructing a rotation angle by which the rotary table 13 should be rotated.

Specifically, in a case where the rotary table 13 formed of an opaque material or a translucent material has the cutout portion 17 as illustrated in FIG. 9, the second motor control signal can be a control signal for rotating the rotary table 13 such that the cutout portion 17 is moved to a position immediately below the opening 11.

In addition, in a case where the rotary table 13 formed of a transparent material includes the detection device 50 formed of an opaque material or a translucent material, the second motor control signal can be a control signal for rotating the rotary table 13 such that the first imaging device 14 and the detection device 50 are moved to positions other than the positions immediately below the opening 11.

Furthermore, in a case where the rotary table 13 and the detection device 50 are formed of a transparent material, the second motor control signal can be a control signal for rotating the rotary table 13 such that the first imaging device 14 is moved to a position other than the position immediately below the opening 11.

FIG. 11 is a flowchart illustrating an example of processing executed by the motor control apparatus 70. In step S1, the size determination unit 101 of the motor control apparatus 70 determines whether or not the size of the finger as the imaging target is equal to or smaller than a predetermined size by using the detection information of the detection device 50.

In a case where the size of the finger as the imaging target is equal to or smaller than the predetermined size (YES), the motor control unit 102 transmits the first motor control signal to the motor in step S2, and the processing of FIG. 11 is ended. When the first motor control signal is received, the motor rotates the rotary table 13 such that the first imaging device 14 is moved to a position immediately below the opening 11.

On the other hand, in a case where the size of the finger as the imaging target exceeds the predetermined size (NO), the motor control unit 102 transmits the second motor control signal to the motor in step S3, and the processing of FIG. 11 is ended. When the second motor control signal is received, the motor rotates the rotary table 13 such that the fingerprint imaging by the second imaging device becomes possible.

As described above, in the third example embodiment, the fingerprint imaging device 1 further includes the detection device 50 that detects the size of the finger as the imaging target placed on the opening 11, the motor that rotates the rotary table 13, and the motor control apparatus 70 that controls the rotation of the motor. The size determination unit 101 of the motor control apparatus 70 determines whether or not the size of the finger as the imaging target is equal to or smaller than a predetermined size by using the detection information of the detection device 50. In a case where the size of the finger as the imaging target is equal to or smaller than the predetermined size, the motor control unit 102 transmits the first motor control signal to the motor. When the first motor control signal is received, the motor rotates the rotary table 13 such that the first imaging device 14 is moved to a position immediately below the opening 11.

Thus, in a case where the size of the finger detected by the detection device 50 is equal to or smaller than the predetermined size, for example, in a case where the size of the finger detected by the detection device 50 is the size of the finger of the newborn baby, the first imaging device 14 is moved to a position immediately below the opening 11, and thus the fingerprint imaging of the newborn baby by the first imaging device 14 becomes possible. Therefore, the imaging devices to be used for imaging a fingerprint can be switched depending on the size of the finger as the imaging target.

In addition, in a case where the size of the finger as the imaging target exceeds the predetermined size, the motor control unit 102 of the motor control apparatus 70 transmits the second motor control signal to the motor. When the second motor control signal is received, the motor rotates the rotary table 13 such that the fingerprint imaging by the second imaging device 15 becomes possible.

Thus, in a case where the size of the finger detected by the detection device 50 exceeds the predetermined size, for example, in a case where the size of the finger detected by the detection device 50 is the size of the finger of the adult, the fingerprint imaging of the adult by the second imaging device 15 becomes possible. Therefore, the imaging devices to be used for imaging a fingerprint can be switched depending on the size of the finger as the imaging target.

### Fourth Example Embodiment

In a fourth example embodiment, the fingerprint imaging device 1 can switch between the first imaging device 14 and the second imaging device 15 to be used for imaging a fingerprint, by the user operating rotation instruction means 80 for rotating the rotary table 13. Hereinafter, differences from the first example embodiment will be mainly described.

FIG. 12 is a plan view illustrating the rotation mechanism of the fingerprint imaging device 1 according to the fourth example embodiment. The fingerprint imaging device 1 further includes the rotation instruction means 80, a gear 82, and a gear 83. Note that the configuration of the rotation mechanism using gears illustrated in FIG. 12 is an example, and various other types of gears may be adopted. In addition, the number of gears to be used is not limited to the configuration illustrated in FIG. 12.

The rotation instruction means 80 can be configured to project from one side surface of the housing 10 in the transverse direction of the fingerprint imaging device 1. The rotation instruction means 80 can be moved in the transverse direction of the fingerprint imaging device 1. The sectional shape of the rotation instruction means 80 can be, for example, a quadrangle. The thickness of the rotation instruction means 80 is equal to or less than a gap formed between the lower surface of the upper portion 10a and the upper surface of the lower portion 10b of the housing 10. The width of the rotation instruction means 80 is a width that does not interfere with the rotary table 13.

As illustrated in FIG. 12, the rotation instruction means 80 has a plurality of protrusions 81 on a side surface on the proximal side of the rotary table 13. The plurality of protrusions 81 of the rotation instruction means 80 engage with the gear 82.

The gear 82 is attached to a strut 84 and can be rotated about the strut 84. The thickness of the gear 82 is a thickness that does not interfere with the lower surface of the upper portion 10a of the housing 10 and the rotary table 13. The strut 84 is attached to the lower surface of the upper portion 10a of the housing 10, and extends from the lower surface of the upper portion 10a of the housing 10 to the lower surface of the gear 82. The gear 82 engages with the gear 83.

The gear 83 is fixed to the upper surface of the rotary table 13, and can be rotated about the strut 12. The thickness of the gear 83 is a thickness that does not interfere with the lower surface of the upper portion 10a of the housing 10.

The size and pitch of the protrusions 81 of the rotation instruction means 80, the size of the gears 82 and 83, and the size and pitch of the teeth of the gears 82 and 83 can be configured such that, in a case where the rotation instruction means 80 is pressed inward in the transverse direction of the fingerprint imaging device 1 so that the exposed area of the rotation instruction means 80 is minimized, for example, in a case where a distal end portion 85 comes into contact with the inner surface of the housing 10, the rotary table 13 is rotated so that the first imaging device 14 is moved to a position immediately below the opening 11.

On the other hand, the protrusions 81 of the rotation instruction means 80, the size of the gears 82 and 83, and the size and pitch of the teeth of the gears 82 and 83 can be configured such that, in a case where the rotation instruction means 80 is pulled outward in the transverse direction from a state where the exposed area of the rotation instruction means 80 is minimized, the fingerprint imaging by the second imaging device 15 becomes possible. The state where the fingerprint imaging by the second imaging device 15 becomes possible is a state where the first imaging device 14 is positioned other than immediately below the opening 11 in a case where the rotary table 13 formed of a transparent material is adopted. In addition, in a case where the rotary table 13 formed of an opaque material or a translucent material is adopted, the state where the fingerprint imaging by the second imaging device 15 becomes possible is a state where the cutout portion 17 of the rotary table 13 is positioned immediately below the opening 11.

As described above, in the fourth example embodiment, the fingerprint imaging device 1 further includes the rotation instruction means 80 for rotating the rotary table 13. The rotation instruction means 80 can be configured to project from one side surface of the housing 10 in the transverse direction of the fingerprint imaging device 1 and to be moved in the transverse direction. The rotation instruction means 80 and the rotary table 13 are connected via the gears 82 and 83. In a case where the rotation instruction means 80 is pressed inward in the transverse direction so that the exposed area of the rotation instruction means 80 is minimized, the rotation instruction means 80 and the gears 82 and 83 rotate the rotary table 13 such that the first imaging device 14 is positioned immediately below the opening 11. In addition, in a case where the rotation instruction means 80 is pulled, from a state where the exposed area of the rotation instruction means 80 is minimized, outward in the transverse direction so that the exposed area of the rotation instruction means 80 is no longer minimum, the rotation instruction means 80 and the gears 82 and 83 rotate the rotary table 13 such that the fingerprint imaging by the second imaging device 15 becomes possible. Thus, the user can switch between the first imaging device 14 and the second imaging device 15 to be used for imaging a fingerprint, by operating the rotation instruction means 80 with one hand.

### Fifth Example Embodiment

In a fifth example embodiment, the fingerprint imaging device 1 can switch between the first imaging device 14 and the second imaging device 15 to be used for imaging a fingerprint, by the user operating the rotation instruction means 80 that projects from two side surfaces of the housing 10. Hereinafter, differences from the fourth example embodiment will be mainly described.

The rotation instruction means 80 can be configured to project from two side surfaces of the housing 10 in the transverse direction of the fingerprint imaging device 1 and to be moved in the transverse direction of the fingerprint imaging device 1.

In a case where the rotation instruction means 80 is pressed from the side of a first side surface of the two side surfaces, the rotation instruction means 80 rotates the rotary table 13 such that the first imaging device 14 is moved to a position immediately below the opening 11. The rotation instruction means 80 and the rotary table 13 can be configured such that, in a case where the user presses the rotation instruction means 80 from the side of the first side surface, the rotary table 13 is rotated so that the first imaging device 14 is moved to a position immediately below the opening 11.

In addition, in a case where the rotation instruction means 80 is pressed from the side of a second side surface different from the first side surface, the rotation instruction means 80 rotates the rotary table 13 such that the fingerprint imaging by the second imaging device 15 becomes possible. The rotation instruction means 80 and the rotary table 13 can be configured such that, in a case where the user presses the rotation instruction means 80 from the side of the second side surface, the rotary table 13 is rotated such that the fingerprint imaging by the second imaging device 15 becomes possible.

Specifically, in a case where the rotary table 13 formed of an opaque material or a translucent material is adopted, the rotation instruction means 80 can be configured such that the cutout portion 17 of the rotary table 13 is positioned immediately below the opening 11 in a state where the exposed area of the rotation instruction means 80 on the side of the second side surface is minimized. In addition, in a case where the rotary table 13 formed of a transparent material is adopted, the rotation instruction means 80 can be configured such that the first imaging device 14 is positioned other than immediately below the opening 11 in a state where the exposed area of the rotation instruction means 80 on the side of the second side surface is minimized.

As described above, in the fifth example embodiment, the fingerprint imaging device 1 further includes the rotation instruction means 80 for rotating the rotary table 13. The rotation instruction means 80 is configured to project from two side surfaces of the housing 10 in the transverse direction of the fingerprint imaging device 1 and to be moved in the transverse direction. The rotation instruction means 80 and the rotary table 13 are connected via the gears 82 and 83. In a case where the rotation instruction means 80 is pressed from the side of the first side surface of the two side surfaces, the rotation instruction means 80 and the gears 82 and 83 rotate the rotary table 13 such that the first imaging device 14 is moved to a position immediately below the opening 11. On the other hand, in a case where the rotation instruction means 80 is pressed from the side of the second side surface different from the first side surface, the rotation instruction means 80 and the gears 82 and 83 rotate the rotary table 13 such that the fingerprint imaging by the second imaging device 15 becomes possible. Thus, the user can switch between the first imaging device 14 and the second imaging device 15 to be used for imaging a fingerprint, by operating the rotation instruction means 80.

### Sixth Example Embodiment

In a sixth example embodiment, the fingerprint imaging device 1 can switch between the first imaging device 14 and the second imaging device 15 to be used for imaging a fingerprint, by the user opening and closing the openable upper portion 10a of the housing 10. Hereinafter, differences from the first example embodiment will be mainly described.

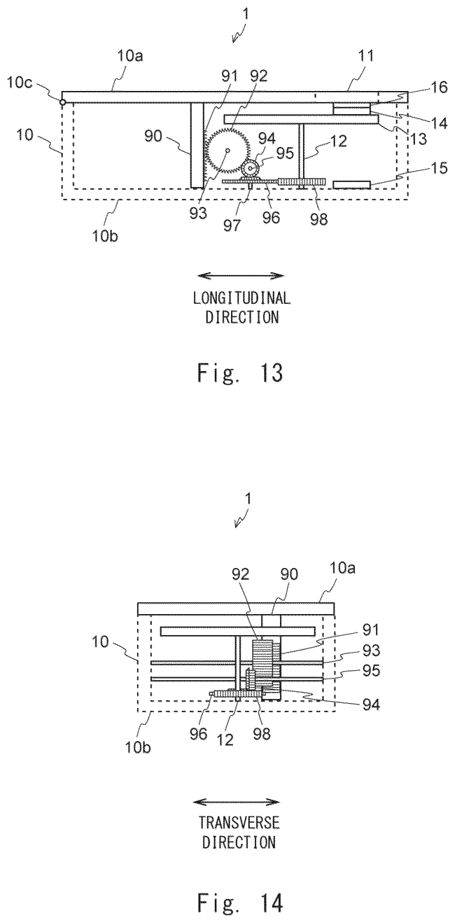

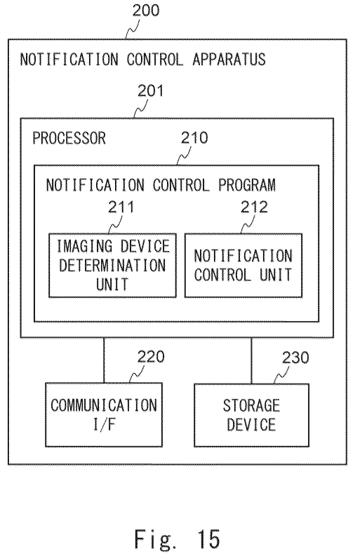

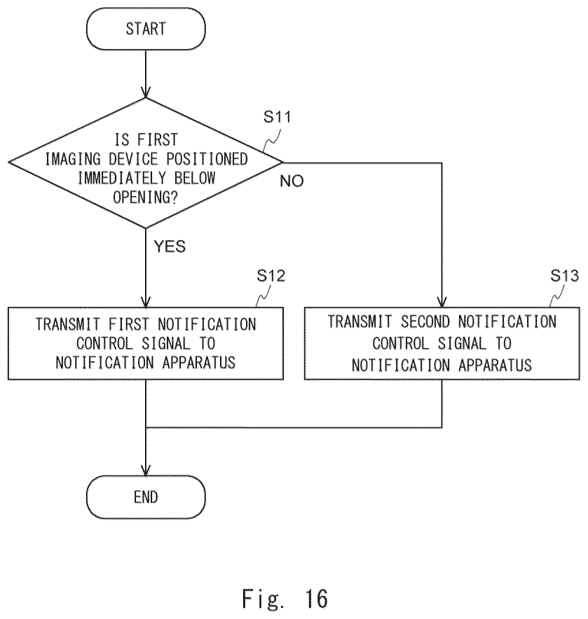

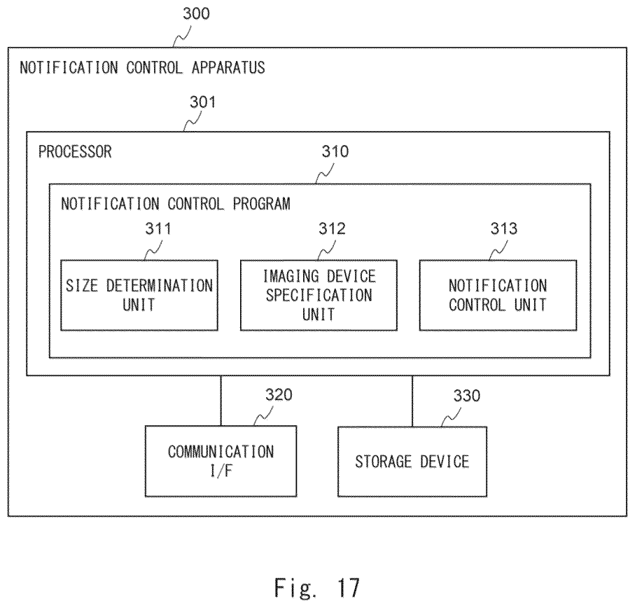

FIG. 13 is an elevation view of the fingerprint imaging device 1 according to the sixth example embodiment as viewed from the transverse direction. FIG. 14 is an elevation view of the fingerprint imaging device 1 according to the sixth example embodiment as viewed from the longitudinal direction. Note that the configuration of the rotation mechanism using gears illustrated in FIGS. 13 and 14 is an example, and various other types of gears may be adopted. In addition, the number of gears to be used is not limited to the configuration illustrated in FIGS. 13 and 14.

As illustrated in FIG. 13, the upper portion 10a of the housing 10 is attached to the housing 10 so as to be rotatable about an end portion 10c of the upper portion 10a of the housing 10 on a side where the opening 11 is not formed. The rotation of the upper portion 10a of the housing 10 can be realized by connection means such as pin joining. The upper portion 10a of the housing 10 is opened and closed by the rotation of the upper portion 10a of the housing 10.

The upper portion 10a of the housing 10 includes rotation instruction means 90 for rotating the rotary table 13. The rotation instruction means 90 is attached to the upper portion 10a of the housing 10 so as to be parallel to the strut 12. As illustrated in FIG. 13, the rotation instruction means 90 has a plurality of protrusions 91 on a side surface on the proximal side of the rotary table 13. The plurality of protrusions 91 engage with a gear 92.

The gear 92 rotates about a strut 93 extending in the transverse direction of the fingerprint imaging device 1. The strut 93 is attached to the inner surface of the housing 10. The gear 92 engages with a spur gear of a composite gear 94, as illustrated in FIG. 14.

The composite gear 94 is a gear formed of a spur gear and a bevel gear, and is rotated about a rotation axis 95. The rotation axis 95 is attached to the inner surface of the housing 10 so as to be parallel to the strut 93. The composite gear 94 engages with a composite gear 96.

The composite gear 96 is a gear formed of a spur gear and a bevel gear, and is rotated about a strut 97. The bevel gear of the composite gear 96 engages with the bevel gear of the composite gear 94. The strut 97 is attached to the lower portion 10b of the housing 10 so as to be parallel to the strut 12. The spur gear of the composite gear 96 engages with a gear 98.

The gear 98 is fixed to the strut 12. In the present example embodiment, the strut 12 is rotatably attached to the lower portion 10b of the housing 10. In addition, the strut 12 extends to the upper surface of the rotary table 13. The rotary table 13 is fixed to the strut 12.

The size and pitch of the protrusions 91 of the rotation instruction means 90, the size of the gears 92, 94, 96, and 98, and the size and pitch of the teeth of the gears 92, 94, 96, and 98 can be configured such that when the upper portion 10a of the housing 10 is closed, the rotary table 13 is rotated so that the first imaging device 14 is moved to a position immediately below the opening 11. The size and pitch of the protrusions 91 of the rotation instruction means 90 and the gears 92, 94, 96, and 98 can be configured such that when the upper portion 10a of the housing 10 is opened, the rotary table 13 is rotated such that the fingerprint imaging by the second imaging device 15 becomes possible.

Specifically, in a case where the rotary table 13 formed of an opaque material or a translucent material is adopted, the size and pitch of the protrusions 91 of the rotation instruction means 90 and the gears 92, 94, 96, and 98 can be configured such that when the upper portion 10a of the housing 10 is opened, the cutout portion 17 of the rotary table 13 is positioned immediately below the opening 11. In addition, in a case where the rotary table 13 formed of a transparent material is adopted, the size and pitch of the protrusions 91 of the rotation instruction means 90 and the gears 92, 94, 96, and 98 can be configured such that when the upper portion 10a of the housing 10 is opened, the first imaging device 14 is positioned other than immediately below the opening 11.

As described above, in the sixth example embodiment, the upper portion 10a of the housing 10 is attached to the housing 10 so as to be rotatable about the end portion 10c of the upper portion 10a of the housing 10 on a side where the opening 11 is not formed. The upper portion 10a of the housing 10 includes rotation instruction means 90 for rotating the rotary table 13. The rotation instruction means 90 is attached to the upper portion 10a so as to be parallel to the strut 12. The rotation instruction means 90 and the rotary table 13 are connected via the gears 92, 94, 96, and 98. When the upper portion 10a is closed, the rotation instruction means 90 and the gears 92, 94, 96, and 98 rotate the rotary table 13 such that the first imaging device 14 is moved to a position immediately below the opening 11. On the other hand, when the upper portion 10a is opened, the rotation instruction means 90 and the gears 92, 94, 96, and 98 rotate the rotary table 13 such that the fingerprint imaging by the second imaging device 15 becomes possible. Thus, the user can switch between the first imaging device 14 and the second imaging device 15 to be used for imaging a fingerprint, by opening and closing the upper portion 10a.

### Seventh Example Embodiment

In a seventh example embodiment, the fingerprint imaging device 1 includes a notification apparatus that provides notification of which one of the fingerprint imaging by the first imaging device 14 and the fingerprint imaging by the second imaging device 15 is possible, and a notification control apparatus 200 that controls the notification apparatus. Hereinafter, differences from the first example embodiment will be mainly described.

Specific examples of the notification apparatus include a lighting device such as an LED and a display device. In a case where the lighting device is adopted as the notification apparatus, the lighting device can be installed at an arbitrary position of the fingerprint imaging device 1, for example, on the upper surface, the lower surface, the outer surface, and the like of the housing 10. In addition, in a case where the display device is adopted as the notification apparatus, the fingerprint imaging device 1 can be configured to include the display device. In this case, the display device can be installed at an arbitrary position of the fingerprint imaging device 1, for example, on the upper surface, the lower surface, the outer surface, and the like of the housing 10. Furthermore, the display device that is a device separate from the fingerprint imaging device 1 can be adopted as the notification apparatus. In this case, the fingerprint imaging device 1 and the display device can communicate with each other in a wired or wireless manner. Examples of the display device as an external device include a monitor provided in a PC or the like.

FIG. 15 is a diagram illustrating a configuration of the notification control apparatus 200 according to the seventh example embodiment. Specific examples of the notification control apparatus 200 include a microcomputer and the like. The notification control apparatus 200 includes a processor 201 capable of executing various programs, a communication interface (I/F) 220, and a storage device 230. Specific examples of the processor 201 include various processors such as a CPU and an MPU.

The notification control apparatus 200 receives information output from another device included in the fingerprint imaging device 1 via the communication interface 220. In addition, the notification control apparatus 200 transmits a control signal for controlling the notification apparatus to the notification apparatus via the communication interface 220.

The storage device 230 stores a notification control program 210 executed by the processor 201 and various kinds of information. The processor 201 executes a method according to the exemplary example embodiment by reading the notification control program 210 from the storage device 230 and executing the notification control program 210. The notification control program 210 includes an imaging device determination unit 211 and a notification control unit 212. Note that the functions of the notification control program 210 may be implemented by an integrated circuit such as FPGA or ASIC. An integrated circuit such as a processor, an FPGA, and an ASIC corresponds to a computer.

The imaging device determination unit 211 is a program that determines whether or not the first imaging device 14 is positioned immediately below the opening 11. For example, the imaging device determination unit 211 can determine whether or not the first imaging device 14 is positioned immediately below the opening 11 on the basis of the luminance of the captured image generated by the first imaging device 14. In a case where the luminance of the captured image generated by the first imaging device 14 is equal to or greater than a predetermined threshold, the imaging device determination unit 211 determines that the first imaging device 14 is positioned immediately below the opening 11. The predetermined threshold can be luminance of an image captured by the first imaging device 14 when the first imaging device 14 is positioned immediately below the opening 11. In the present example embodiment, the housing 10 is preferably not formed of a transparent material.

In addition, the imaging device determination unit 211 can determine whether or not the first imaging device 14 is positioned immediately below the opening 11 on the basis of the rotation angle of the rotary table 13. In this case, the fingerprint imaging device 1 includes an angle sensor that detects the rotation angle of the rotary table 13. In a case where the rotation angle output from the angle sensor is a predetermined angle, the imaging device determination unit 211 determines that the first imaging device 14 is positioned immediately below the opening 11. The predetermined angle is an angle output by the angle sensor when the first imaging device 14 is positioned immediately below the opening 11.

The notification control unit 212 is a program that controls the notification apparatus according to the determination result of the imaging device determination unit 211. In a case where it is determined that the first imaging device 14 is positioned immediately below the opening 11, the notification control unit 212 transmits a first notification control signal to the notification apparatus. The first notification control signal according to the present example embodiment is a signal for causing the notification apparatus to provide notification that the fingerprint imaging by the first imaging device 14 is possible. For example, in a case where the lighting device is adopted as the notification apparatus, the lighting device can provide notification that the fingerprint imaging by the first imaging device 14 is possible, by using a lighting pattern or a lighting color. For example, in a case where the display device is adopted as the notification apparatus, the display device can display an image indicating that the fingerprint imaging by the first imaging device 14 is possible on a screen of the display device.

On the other hand, in a case where it is determined that the first imaging device 14 is not positioned immediately below the opening 11, the notification control unit 212 transmits a second notification control signal to the notification apparatus. The second notification control signal according to the present example embodiment is a signal for causing the notification apparatus to provide notification that the fingerprint imaging by the second imaging device 15 is possible. For example, in a case where the lighting device is adopted as the notification apparatus, the lighting device can provide notification that the fingerprint imaging by the second imaging device 15 is possible, by using a lighting pattern or a lighting color different from the lighting pattern or the lighting color based on the first notification control signal. In addition, for example, in a case where the display device is adopted as the notification apparatus, the display device can display an image indicating that the fingerprint imaging by the second imaging device 15 is possible on a screen of the display device.

FIG. 16 is a flowchart illustrating an example of processing executed by the notification control apparatus 200. In step S11, the imaging device determination unit 211 of the notification control apparatus 200 determines whether or not the first imaging device 14 is positioned immediately below the opening 11.

In a case where the first imaging device 14 is positioned immediately below the opening 11 (YES), the notification control unit 212 transmits the first notification control signal to the notification apparatus in step S12, and the processing of FIG. 16 is ended. 1. When the first notification control signal is received, the notification apparatus provides notification that the fingerprint imaging by the first imaging device 14 is possible.

On the other hand, in a case where the first imaging device 14 is not positioned immediately below the opening 11 (NO), the notification control unit 212 transmits the second notification control signal to the notification apparatus in step S13, and the processing of FIG. 16 is ended. When the second notification control signal is received, the notification apparatus provides notification that the fingerprint imaging by the second imaging device 15 is possible.

As described above, in the seventh example embodiment, the fingerprint imaging device 1 includes the notification apparatus that provides notification of which of the fingerprint imaging by the first imaging device 14 and the fingerprint imaging by the second imaging device 15 is possible, and the notification control apparatus 200 that controls the notification apparatus. The imaging device determination unit 211 of the notification control apparatus 200 determines whether or not the first imaging device 14 is positioned immediately below the opening 11 on the basis of the luminance of the captured image generated by the first imaging device 14 or the rotation angle of the rotary table 13. In a case where it is determined that the first imaging device 14 is positioned immediately below the opening 11, the notification control unit 212 causes the notification apparatus to provide notification that the fingerprint imaging by the first imaging device 14 is possible. On the other hand, in a case where it is determined that the first imaging device 14 is not positioned immediately below the opening 11, the notification control unit 212 causes the notification apparatus to provide notification that the fingerprint imaging by the second imaging device 15 is possible. As a result, the user of the fingerprint imaging device 1 can ascertain which one of the fingerprint imaging by the first imaging device 14 and the fingerprint imaging by the second imaging device 15 is possible, on the basis of the notification by the notification apparatus.

### Eighth Example Embodiment

In an eighth example embodiment, the fingerprint imaging device 1 includes the notification apparatus that provides notification of whether or not the correspondence between the finger placed on the opening 11 and the first imaging device 14 and the second imaging device 15 is correct, and a notification control apparatus 300 that controls the notification apparatus. Hereinafter, differences from the seventh example embodiment will be mainly described.

FIG. 17 is a diagram illustrating a configuration of the notification control apparatus 300 according to the eighth example embodiment. Specific examples of the notification control apparatus 300 include a microcomputer and the like. The notification control apparatus 300 includes a processor 301 capable of executing various programs, a communication interface (I/F) 320, and a storage device 330. Specific examples of the processor 301 include various processors such as a CPU and an MPU.

The notification control apparatus 300 receives information output from another device included in the fingerprint imaging device 1 via the communication interface 320. In addition, the notification control apparatus 300 transmits a control signal for controlling the notification apparatus to the notification apparatus via the communication interface 320. Specific examples of the notification apparatus include a lighting device and a display device as in the seventh example embodiment.

The storage device 330 stores the motor control program 100 executed by the processor 301 and various kinds of information. The processor 301 executes a method according to the exemplary example embodiment by reading the notification control program 310 from the storage device 330 and executing the notification control program 310. The notification control program 310 includes a size determination unit 311, an imaging device specification unit 312, and a notification control unit 313. Note that the functions of the notification control program 310 may be implemented by an integrated circuit such as FPGA or ASIC. An integrated circuit such as a processor, an FPGA, and an ASIC corresponds to a computer.

The size determination unit 311 determines whether or not the size of the finger included in the captured image generated by the first imaging device 14 or the second imaging device 15 is equal to or smaller than a predetermined size. The predetermined size can be a typical finger size of a newborn baby.

The imaging device specification unit 312 is a program that specifies the imaging device that has imaged the finger as the imaging target placed on the opening 11. Specifically, the imaging device specification unit 312 specifies the imaging device that has imaged the finger as the imaging target by specifying the imaging device that has output the captured image used for the determination by the size determination unit 311. The first imaging device 14 and the second imaging device 15 add identification information of the first imaging device 14 and the second imaging device 15 to the captured image. The imaging device specification unit 312 can specify the imaging device that has output the captured image used for the determination by the size determination unit 311, on the basis of the identification information of the imaging device added to the captured image.

The notification control unit 313 is a program that controls the notification apparatus according to the determination result of the size determination unit 311 and the imaging device specification unit 312. In a case where the correspondence between the finger placed on the opening 11 and the imaging device is correct, the notification control unit 313 transmits the first notification control signal to the notification apparatus. The first notification control signal according to the present example embodiment is a signal for causing the notification apparatus to provide notification that the correspondence between the finger placed on the opening 11 and the imaging device is correct. For example, in a case where the lighting device is adopted as the notification apparatus, the lighting device can provide notification that the correspondence between the finger placed on the opening 11 and the imaging device is correct, by using a lighting pattern or a lighting color. In addition, in a case where the display device is adopted as the notification apparatus, the display device can display an image indicating that the correspondence between the finger placed on the opening 11 and the imaging device is correct, on a screen of the display device.

On the other hand, in a case where the correspondence between the finger placed on the opening 11 and the imaging device is incorrect, the notification control unit 212 transmits the second notification control signal to the notification apparatus. The second notification control signal according to the present example embodiment is a signal for causing the notification apparatus to provide notification that the correspondence between the finger placed on the opening 11 and the imaging device is incorrect. For example, in a case where the lighting device is adopted as the notification apparatus, the lighting device can provide notification that the correspondence between the finger placed on the opening 11 and the imaging device is incorrect, by using a lighting pattern or a lighting color different from the lighting pattern or the lighting color based on the first notification control signal. In addition, in a case where the display device is adopted as the notification apparatus, the display device can display an image indicating that the correspondence between the finger placed on the opening 11 and the imaging device is incorrect, on a screen of the display device.

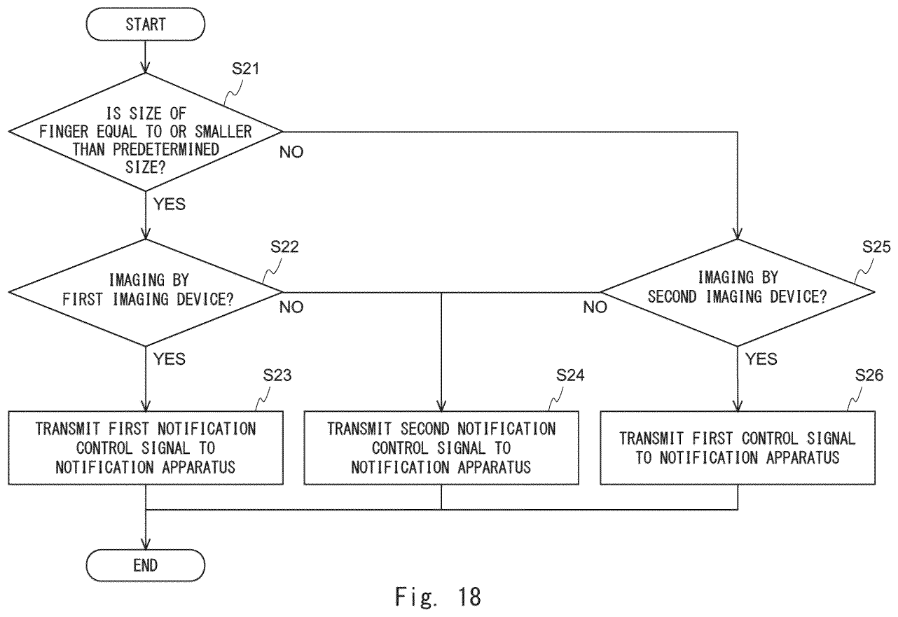

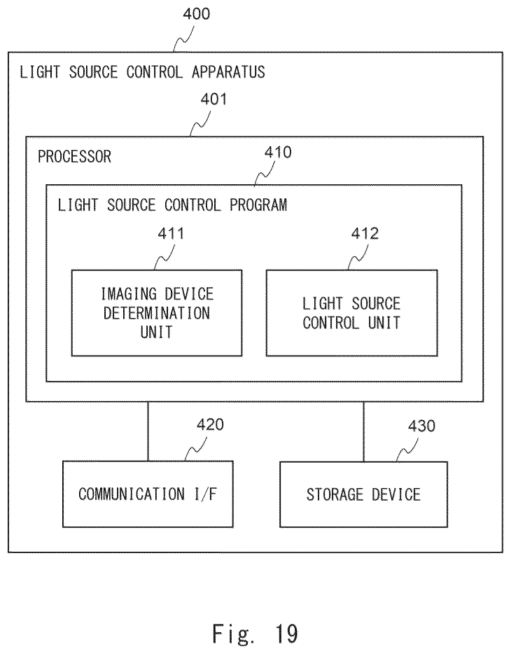

FIG. 18 is a flowchart illustrating an example of processing executed by the notification control apparatus 300. In step S21, the size determination unit 311 of the notification control apparatus 300 determines whether or not the size of the finger included in the captured image generated by the imaging device is equal to or smaller than a predetermined size. In a case where the size of the finger is equal to or smaller than the predetermined size (YES), the imaging device specification unit 312 determines whether or not the imaging device that has imaged the finger placed on the opening 11 is the first imaging device 14, in step S22.

In a case where the imaging device that has imaged the finger is the first imaging device 14 (YES), the notification control unit 212 transmits the first notification control signal to the notification apparatus in step S23, and the processing of FIG. 18 is ended. When the first notification control signal is received, the notification apparatus provides notification that the correspondence between the finger placed on the opening 11 and the imaging device is correct.

On the other hand, in a case where it is determined in step S22 that the imaging device that has imaged the finger is not the first imaging device 14, that is, that imaging device is the second imaging device 15 (NO), the notification control unit 212 transmits the second notification control signal to the notification apparatus in step S24, and the processing of FIG. 18 is ended. When the second notification control signal is received, the notification apparatus provides notification that the correspondence between the finger placed on the opening 11 and the imaging device is incorrect.

In a case where it is determined in step S21 that the size of the finger exceeds the predetermined size (NO), the imaging device determination unit 211 determines whether or not the imaging device that has imaged the finger placed on the opening 11 is the second imaging device 15, in step S25. In a case where the imaging device that has imaged the finger is the second imaging device 15 (YES), the notification control unit 212 transmits the first notification control signal to the notification apparatus in step S26, and the processing of FIG. 18 is ended. When the first notification control signal is received, the notification apparatus provides notification that the correspondence between the finger placed on the opening 11 and the imaging device is correct.

On the other hand, in a case where it is determined in step S25 that the imaging device that has imaged the finger is not the second imaging device 15, that is, that imaging device is the first imaging device 14 (NO), the notification control unit 212 transmits the second notification control signal to the notification apparatus in step S24, and the processing of FIG. 18 is ended. When the second notification control signal is received, the notification apparatus provides notification that the correspondence between the finger placed on the opening 11 and the imaging device is incorrect.

As described above, in the eighth example embodiment, the fingerprint imaging device 1 includes the notification apparatus that provides notification of whether or not the correspondence between the finger as the imaging target placed on the opening 11 and the first imaging device 14 and the second imaging device 15 is correct, and the notification control apparatus 300 that controls the notification apparatus. The size determination unit 311 of the notification control apparatus 300 determines whether or not the size of the finger as the imaging target is equal to or smaller than a predetermined size, by using the captured image generated by the first imaging device 14 or the second imaging device 15. The imaging device specification unit 312 specifies the imaging device that has imaged the finger as the imaging target on the basis of the captured image used for the determination by the size determination unit 311. The notification control unit 313 controls the notification apparatus on the basis of the determination result of the size determination unit 311 and the imaging device specification unit 312.

Specifically, in a case where the size of the finger as the imaging target is equal to or smaller than the predetermined size and the imaging device that has imaged the finger as the imaging target is the first imaging device, the notification control unit 313 causes the notification apparatus to provide notification that the correspondence between the finger placed on the opening 11 and the imaging device is correct. In addition, in a case where the size of the finger as the imaging target exceeds the predetermined size and the imaging device that has imaged the finger as the imaging target is the second imaging device, the notification control unit 313 causes the notification apparatus to provide notification of that the correspondence between the finger placed on the opening 11 and the imaging device is correct.

On the other hand, in a case where the size of the finger as the imaging target is equal to or smaller than the predetermined size and the imaging device that has imaged the finger as the imaging target is the second imaging device, the notification control unit 313 causes the notification apparatus to provide notification that the correspondence between the finger placed on the opening 11 and the imaging device is incorrect. In addition, in a case where the size of the finger as the imaging target exceeds the predetermined size and the imaging device that has imaged the finger as the imaging target is the first imaging device, the notification control unit 313 causes the notification apparatus to provide notification that the correspondence between the finger placed on the opening 11 and the imaging device is incorrect.

Thus, the user of the fingerprint imaging device 1 can ascertain whether or not the correspondence between the finger placed on the opening 11 of the fingerprint imaging device 1 and the currently set imaging device is correct.

### Ninth Example Embodiment

In a ninth example embodiment, the fingerprint imaging device 1 includes a first light source and a second light source that irradiate the finger placed on the opening 11, and a light source control apparatus 400 that controls the first light source and the second light source. Hereinafter, differences from the first example embodiment will be mainly described.

The first light source is used in a case where the fingerprint imaging is performed by the first imaging device 14. The second light source is used in a case where the fingerprint imaging is performed by the second imaging device 15. In the present example embodiment, the first imaging device 14 is used for imaging a fingerprint of a newborn baby. In addition, the second imaging device 15 is used for imaging a fingerprint of a target person other than a newborn baby. The light amount of the second light source is greater than the light amount of the first light source.

The first light source and the second light source are disposed in the housing 10. For example, the first light source and the second light source can be disposed on a side surface forming the opening 11 in the upper portion 10a of the housing 10. In addition, the first light source can be disposed on the rotary table 13 to be adjacent to the first imaging device 14. Furthermore, the second light source can be disposed on the lower surface in the housing 10 to be adjacent to the second imaging device 15.

The wavelengths of the light emitted from the first light source and the second light source can be, for example, 850 nm. Note that the wavelengths of the light emitted from the first light source and the second light source are not limited thereto, and any wavelength can be adopted.

FIG. 19 is a diagram illustrating a configuration of the light source control apparatus 400 according to the ninth example embodiment. Specific examples of the light source control apparatus 400 include a microcomputer and the like. The light source control apparatus 400 includes a processor 401 capable of executing various programs, a communication interface (I/F) 420, and a storage device 430. Specific examples of the processor 401 include various processors such as a CPU and an MPU.

The light source control apparatus 400 receives information output from another device included in the fingerprint imaging device 1 via the communication interface 420. In addition, the light source control apparatus 400 transmits a control signal for controlling the first light source and the second light source to the notification apparatus via the communication interface 420.

The storage device 430 stores a light source control program 410 executed by the processor 401 and various kinds of information. The processor 401 executes a method according to the exemplary example embodiment by reading the light source control program 410 from the storage device 430 and executing the light source control program 410. The light source control program 410 includes an imaging device determination unit 411 and a light source control unit 412. Note that the functions of the light source control program 410 may be implemented by an integrated circuit such as FPGA or ASIC. An integrated circuit such as a processor, an FPGA, and an ASIC corresponds to a computer.

The imaging device determination unit 411 is a program that determines whether or not the first imaging device 14 is positioned immediately below the opening 11. For example, the imaging device determination unit 411 can determine whether or not the first imaging device 14 is positioned immediately below the opening 11 on the basis of the luminance of the captured image generated by the first imaging device 14. In a case where the luminance of the captured image generated by the first imaging device 14 is equal to or greater than a predetermined threshold, the imaging device determination unit 411 determines that the first imaging device 14 is positioned immediately below the opening 11. The predetermined threshold can be luminance of an image captured by the first imaging device 14 when the first imaging device 14 is positioned immediately below the opening 11. In the present example embodiment, the housing 10 is preferably not formed of a transparent material.

In addition, the imaging device determination unit 411 can determine whether or not the first imaging device 14 is positioned immediately below the opening 11 on the basis of the rotation angle of the rotary table 13. In this case, the fingerprint imaging device 1 includes the angle sensor that detects the rotation angle of the rotary table 13. In a case where the rotation angle output from the angle sensor is a predetermined angle, the imaging device determination unit 411 determines that the first imaging device 14 is positioned immediately below the opening 11. The predetermined angle is an angle output by the angle sensor when the first imaging device 14 is positioned immediately below the opening 11.

The light source control unit 412 is a program that controls the first light source and the second light source according to the determination result of the imaging device determination unit 411. In a case where it is determined that the first imaging device 14 is positioned immediately below the opening 11, the light source control unit 412 turns on the first light source. On the other hand, in a case where it is determined that the first imaging device 14 is not positioned immediately below the opening 11, the light source control unit 412 turns on the second light source.

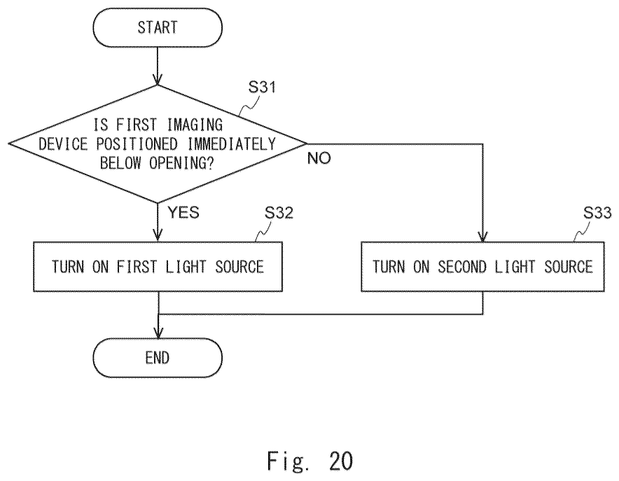

FIG. 20 is a flowchart illustrating an example of processing executed by the light source control apparatus 400. In step S31, the imaging device determination unit 411 of the light source control apparatus 400 determines whether or not the first imaging device 14 is positioned immediately below the opening 11.

In a case where the first imaging device 14 is positioned immediately below the opening 11 (YES), the light source control unit 412 turns on the first light source in step S32, and the processing of FIG. 20 is ended. On the other hand, in a case where the first imaging device 14 is not positioned immediately below the opening 11 (NO), the light source control unit 412 turns on the second light source in step S33, and the processing of FIG. 20 is ended.

As described above, in the ninth example embodiment, the fingerprint imaging device 1 includes the first light source and the second light source that irradiate the finger placed on the opening 11, and the light source control apparatus 400 that controls the first light source and the second light source. The imaging device determination unit 411 of the light source control apparatus 400 determines whether or not the first imaging device 14 is positioned immediately below the opening 11 on the basis of the luminance of the captured image generated by the first imaging device 14 or the rotation angle of the rotary table 13. In a case where it is determined that the first imaging device 14 is positioned immediately below the opening 11, the light source control unit 412 turns on the first light source. On the other hand, in a case where it is determined that the first imaging device 14 is not positioned immediately below the opening 11, the light source control unit 412 turns on the second light source that emits a light amount greater than the light amount of the first light source.

Thus, the light source can be switched according to the imaging device that performs the fingerprint imaging. In a case where the fingerprint imaging of a target person other than a newborn baby, for example, an adult is performed using the second imaging device 15, the second light source that emits a light amount greater than the light amount of the first light source is turned on. Therefore, since the finger of an adult of which the finger is thicker than the finger of a newborn baby is irradiated with a large amount of light, a clear fingerprint image can be obtained.

### Tenth Example Embodiment

In a tenth example embodiment, the fingerprint imaging device 1 includes one light source that irradiates the finger placed on the opening 11, and the light source control apparatus 400 that controls the light source. Hereinafter, differences from the ninth example embodiment will be mainly described.

The light source is a dimming light source capable of configuring a light amount. The light source is disposed in the housing 10. For example, the light source can be disposed on a side surface forming the opening 11 in the upper portion 10a of the housing 10. The wavelength of the light emitted from the light source can be, for example, 850 nm. Note that the wavelength of the light emitted from the light source is not limited thereto, and any wavelength can be adopted.

The light source control unit 412 according to the present example embodiment is a program that controls the light source according to the determination result of the imaging device determination unit 411. In a case where it is determined that the first imaging device 14 is positioned immediately below the opening 11, the light source control unit 412 turns on the light source with a predetermined light amount. The predetermined light amount is a light amount suitable for performing the fingerprint imaging of a newborn baby by the first imaging device 14. On the other hand, in a case where it is determined that the first imaging device 14 is not positioned immediately below the opening 11, the light source control unit 412 turns on the light source with a light amount greater than the predetermined light amount.

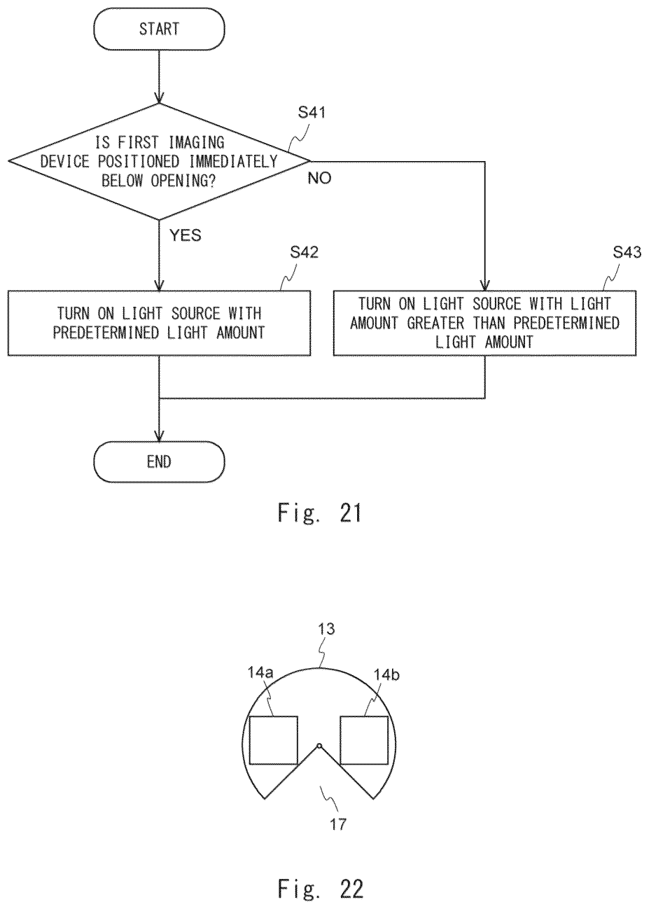

FIG. 21 is a flowchart illustrating an example of processing executed by the light source control apparatus 400. In step S41, the imaging device determination unit 411 of the light source control apparatus 400 determines whether or not the first imaging device 14 is positioned immediately below the opening 11.

In a case where the first imaging device 14 is positioned immediately below the opening 11 (YES), the light source control unit 412 turns on the light source with a predetermined light amount in step S42, and the processing of FIG. 21 is ended. On the other hand, in a case where the first imaging device 14 is not positioned immediately below the opening 11 (NO), the light source control unit 412 turns on the light source with a light amount greater than the predetermined light amount in step S43, and the processing of FIG. 21 is ended.

As described above, in the tenth example embodiment, the fingerprint imaging device 1 includes one light source that irradiates the finger placed on the opening 11, and the light source control apparatus 400 that controls the light source. The imaging device determination unit 411 of the light source control apparatus 400 determines whether or not the first imaging device 14 is positioned immediately below the opening 11 on the basis of the luminance of the captured image generated by the first imaging device 14 or the rotation angle of the rotary table 13. In a case where it is determined that the first imaging device 14 is positioned immediately below the opening 11, the light source control unit 412 turns on the light source with a predetermined light amount. On the other hand, in a case where it is determined that the first imaging device 14 is not positioned immediately below the opening 11, the light source control unit 412 turns on the light source with a light amount greater than the predetermined light amount.

Thus, the light amount can be changed according to the imaging device that performs the fingerprint imaging. In a case where the fingerprint imaging of a target person other than a newborn baby, for example, an adult is performed using the second imaging device 15, the second light source that emits a light amount greater than the light amount of the first light source is turned on. Therefore, since the finger of an adult of which the finger is thicker than the finger of a newborn baby is irradiated with a large amount of light, a clear fingerprint image can be obtained.

### Eleventh Example Embodiment

In the eleventh example embodiment, the fingerprint imaging device 1 includes a plurality of first imaging devices. Hereinafter, differences from the first example embodiment will be mainly described.

Each of the plurality of first imaging devices 14 is disposed at a different position on the upper surface of the rotary table 13. Each imaging device 14 is moved to a position immediately below the opening 11 as the rotary table 13 is rotated.

FIG. 22 is a view illustrating the upper surface of the rotary table 13 according to the eleventh example embodiment. In the example illustrated in FIG. 22, two first imaging devices 14a and 14b are disposed on the rotary table 13. The first imaging devices 14a and 14b are moved to a position immediately below the opening 11 as the rotary table 13 is rotated. The first imaging devices 14a and 14b positioned immediately below the opening 11 perform the fingerprint imaging. On the other hand, in a case where the first imaging devices 14a and 14b are not positioned immediately below the opening 11, the second imaging device 15 performs the fingerprint imaging. Thus, the user can switch between the plurality of first imaging devices and the second imaging device to perform the fingerprint imaging.

### Twelfth Example Embodiment

In the eleventh example embodiment, the fingerprint imaging device 1 includes the plurality of first imaging devices 14 and one second imaging device 15. In a twelfth example embodiment, the fingerprint imaging device 1 may not include the second imaging device. In the twelfth example embodiment, as in the eleventh example embodiment, each of the plurality of first imaging devices 14 is disposed at a different position on the upper surface of the rotary table 13. Each imaging device 14 is moved to a position immediately below the opening 11 as the rotary table 13 is rotated.

At least one of the plurality of first imaging devices can be an imaging device for imaging a fingerprint of a newborn baby. On the other hand, the other first imaging devices can be imaging devices for imaging a fingerprint of a target person other than a newborn baby. The resolution of the first imaging device that images a fingerprint of a newborn baby is preferably higher than the resolution of the other first imaging devices.

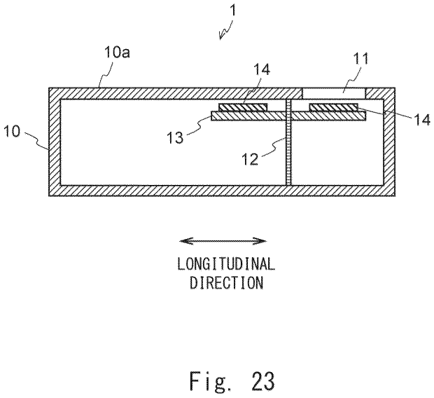

FIG. 23 is a sectional view illustrating main components of the fingerprint imaging device 1 according to the twelfth example embodiment. The fingerprint imaging device 1 includes the housing 10, the strut 12, the rotary table 13, and the plurality of first imaging devices 14.

In the twelfth example embodiment, the fingerprint imaging device 1 includes the plurality of first imaging devices 14 disposed on the rotary table 13. Thus, the user can switch between the plurality of first imaging devices to perform the fingerprint imaging, by rotating the rotary table 13.

In addition, at least one of the plurality of first imaging devices is an imaging device for imaging a fingerprint of a newborn baby. The other first imaging device(s) is imaging device(s) for imaging a fingerprint of a target person other than a newborn baby. The resolution of the imaging device for imaging a fingerprint of a newborn baby can be higher than the resolution of the other first imaging device(s). Thus, a fingerprint image with a high resolution of a newborn baby can be obtained.

In the above-described example, the program includes a group of instructions (or software code) for causing a computer to perform one or more functions described in the example embodiments when being read by the computer. The program may be stored in a non-transitory computer-readable medium or a tangible storage medium. As an example and not by way of limitation, the computer readable medium or the tangible storage medium includes random-access memory (RAM), read-only memory (ROM), a flash memory, a solid-state drive (SSD) or any other memory technology, a CD-ROM, a digital versatile disk (DVD), a Blu-ray (registered trademark) disc or any other optical disk storage, a magnetic cassette, a magnetic tape, a magnetic disk storage, and any other magnetic storage device. The program may be transmitted on a transitory computer-readable medium or a communications medium. By way of example, and not limitation, transitory computer-readable or communication media include electrical, optical, acoustic, or other forms of propagated signals.

The present disclosure is not limited to the above-described example embodiments, and can be appropriately changed without departing from the gist of the present disclosure.

Some or all of the above example embodiments may be described as the following Supplementary Notes, but are not limited to the following.

### Supplementary Note 1

A fingerprint imaging device comprising:
a housing that has an upper portion in which an opening is formed:
a strut that is attached to the housing to be perpendicular to the upper portion of the housing:
a rotary table that is rotatably attached to the strut with the strut as a rotation axis:
a first imaging device that is disposed on an upper surface of the rotary table; and
a second imaging device that is disposed in the housing to be immediately below the opening and below the rotary table, wherein
a part of the rotary table is exposed from the housing, and
the first imaging device is moved to a position immediately below the opening as the rotary table is rotated.

### Supplementary Note 2

The fingerprint imaging device according to Supplementary Note 1, wherein
the first imaging device is an imaging device for imaging a fingerprint of a newborn baby,
the second imaging device is an imaging device for imaging a fingerprint of a target person other than a newborn baby, and
a resolution of the first imaging device is higher than a resolution of the second imaging device.

### Supplementary Note 3

The fingerprint imaging device according to Supplementary Note 1, wherein
the first imaging device and the second imaging device are imaging devices for imaging a fingerprint of a target person other than a newborn baby, and
a resolution of the first imaging device and a resolution of the second imaging device are the same.

### Supplementary Note 4

The fingerprint imaging device according to Supplementary Note 1 or 2, comprising:
a guide disposed on an upper surface of the housing to slide in a longitudinal direction of the fingerprint imaging device, wherein
the guide has a cutout portion formed over an upper surface and a lower surface of the guide, and
the cutout portion is formed such that a size of the cutout portion when viewed from an upper surface side of the guide is equal to or greater than a size of the opening.

### Supplementary Note 5

The fingerprint imaging device according to Supplementary Note 4, wherein
the rotary table is configured to be rotated in conjunction with sliding of the guide,
in a case where the guide is moved to a position where the cutout portion of the guide and the opening are continuous, the guide rotates the rotary table such that the first imaging device is moved to a position immediately below the opening, and
in a case where the guide at the position where the cutout portion of the guide and the opening are continuous is moved away from the opening, the guide rotates the rotary table such that fingerprint imaging of an imaging target by the second imaging device becomes possible.

### Supplementary Note 6

The fingerprint imaging device according to Supplementary Note 1 or 2, comprising:
a detection device that detects a size of a finger as an imaging target placed on the opening:
a motor that rotates the rotary table: and
a motor control apparatus that controls rotation of the motor, wherein
the motor control apparatus includes
size determination means for determining whether or not the size of the finger as the imaging target is equal to or smaller than a predetermined size, by using information detected by the detection device, and
motor control means for controlling the motor,
the motor control means transmits a first motor control signal to the motor in a case where the size of the finger as the imaging target is equal to or smaller than the predetermined size, and
when the motor receives the first motor control signal, the motor rotates the rotary table such that the first imaging device is moved to a position immediately below the opening.

### Supplementary Note 7

The fingerprint imaging device according to Supplementary Note 6, wherein
the motor control means transmits a second motor control signal to the motor in a case where the size of the finger as the imaging target exceeds the predetermined size, and
when the motor receives the second motor control signal, the motor rotates the rotary table such that fingerprint imaging of the imaging target by the second imaging device becomes possible.

### Supplementary Note 8

The fingerprint imaging device according to Supplementary Note 1 or 2, further comprising: rotation instruction means for rotating the rotary table, wherein
the rotation instruction means is configured to project from one side surface of the housing in a transverse direction of the fingerprint imaging device and to be movable in the transverse direction,
the rotation instruction means and the rotary table are connected via one or more gears, and
the rotation instruction means and the gears
rotate the rotary table such that the first imaging device is positioned immediately below the opening in a case where the rotation instruction means is pressed inward in the transverse direction of the fingerprint imaging device so that an exposed area of the rotation instruction means is minimized, and
rotate the rotary table such that fingerprint imaging by the second imaging device becomes possible in a case where the rotation instruction means is pulled outward in the transverse direction of the fingerprint imaging device from a state where the exposed area of the rotation instruction means is minimized and the exposed area of the rotation instruction means is not minimized.

### Supplementary Note 9

The fingerprint imaging device according to Supplementary Note 1 or 2, further comprising: rotation instruction means for rotating the rotary table, wherein
the rotation instruction means is disposed to project from two side surfaces of the housing in a transverse direction of the fingerprint imaging device, and is movable in the transverse direction,
the rotation instruction means and the rotary table are connected via one or more gears, and
the rotation instruction means and the gears
rotate the rotary table such that the first imaging device is moved to a position immediately below the opening in a case where the rotation instruction means is pressed from a side of a first side surface of the two side surfaces, and
rotate the rotary table such that fingerprint imaging by the second imaging device becomes possible in a case where the rotation instruction means is pressed from a side of a second side surface different from the first side surface.

### Supplementary Note 10

The fingerprint imaging device according to Supplementary Note 1 or 2, wherein
the upper portion of the housing is attached to the housing to be rotatable about an end portion of the upper portion of the housing, on a side where the opening is not formed,
the upper portion of the housing includes rotation instruction means for rotating the rotary table,
the rotation instruction means is attached to the upper portion to be parallel to the strut,
the rotation instruction means and the rotary table are connected via one or more gears, and
the rotation instruction means and the gears
rotate the rotary table such that the first imaging device is moved to a position immediately below the opening in a case where the upper portion is closed, and
rotate the rotary table such that fingerprint imaging by the second imaging device becomes possible in a case where the upper portion is opened.

### Supplementary Note 11

The fingerprint imaging device according to Supplementary Note 1 or 2, comprising:
a notification apparatus that provides notification of which one of fingerprint imaging by the first imaging device and fingerprint imaging by the second imaging device is possible: and
a notification control apparatus that controls the notification apparatus, wherein
the notification control apparatus includes
imaging device determination means for determining whether or not the first imaging device is positioned immediately below the opening on the basis of luminance of a captured image generated by the first imaging device or a rotation angle of the rotary table,
notification control means for controlling the notification apparatus on the basis of a determination result of the imaging device determination means, and
the notification control means
causes the notification apparatus to provide notification that fingerprint imaging by the first imaging device is possible in a case where it is determined that the first imaging device is positioned immediately below the opening, and
causes the notification apparatus to provide notification that fingerprint imaging by the second imaging device is possible in a case where it is determined that the first imaging device is not positioned immediately below the opening.

### Supplementary Note 12

The fingerprint imaging device according to Supplementary Note 1 or 2, comprising:
a notification apparatus that provides notification of whether or not correspondence between a finger as an imaging target placed on the opening and the first imaging device and the second imaging device is correct: and
a notification control apparatus that controls the notification apparatus, wherein
the notification control apparatus includes
size determination means for determining whether or not a size of the finger as the imaging target is equal to or smaller than a predetermined size, by using a captured image generated by the first imaging device or the second imaging device,
imaging device specification means for specifying the imaging device that has imaged the finger as the imaging target, on the basis of the captured image used for determination by the size determination means, and
notification control means for controlling the notification apparatus on the basis of determination results of the size determination means and the imaging device specification means, and
the notification control means
causes the notification apparatus to provide notification that the correspondence between the finger as the imaging target and the imaging device is correct in a case where the size of the finger as the imaging target is equal to or smaller than the predetermined size and the imaging device that has imaged the finger as the imaging target is the first imaging device or in a case where the size of the finger as the imaging target exceeds the predetermined size and the imaging device that has imaged the finger as the imaging target is the second imaging device, and
causes the notification apparatus to provide notification that the correspondence between the finger as the imaging target and the imaging device is incorrect in a case where the size of the finger as the imaging target is equal to or smaller than the predetermined size and the imaging device that has imaged the finger as the imaging target is the second imaging device or in a case where the size of the finger as the imaging target exceeds the predetermined size and the imaging device that has imaged the finger as the imaging target is the first imaging device.

### Supplementary Note 13

The fingerprint imaging device according to Supplementary Note 1 or 2, comprising:
a first light source and a second light source that irradiate a finger as an imaging target placed on the opening: and
a light source control apparatus that controls the first light source and the second light source, wherein
a light amount of the second light source is greater than a light amount of the first light source,
the light source control apparatus includes
imaging device determination means for determining whether or not the first imaging device is positioned immediately below the opening on the basis of luminance of a captured image generated by the first imaging device or a rotation angle of the rotary table, and
light source control means for controlling the first light source and the second light source on the basis of a determination result of the imaging device determination means, and
the light source control means
turns on the first light source in a case where it is determined that the first imaging device is positioned immediately below the opening, and
turns on the second light source in a case where it is determined that the first imaging device is not positioned immediately below the opening.

### Supplementary Note 14

The fingerprint imaging device according to Supplementary Note 1 or 2, comprising:
one light source that irradiates a finger as an imaging target placed on the opening: and
a light source control apparatus that controls the light source, wherein
the light source control apparatus includes
imaging device determination means for determining whether or not the first imaging device is positioned immediately below the opening on the basis of luminance of a captured image generated by the first imaging device or a rotation angle of the rotary table, and
light source control means for controlling the light source on the basis of a determination result of the imaging device determination means, and
the light source control means
turns on the light source with a predetermined light amount in a case where it is determined that the first imaging device is positioned immediately below the opening, and
turns on the light source with a light amount greater than the predetermined light amount in a case where it is determined that the first imaging device is not positioned immediately below the opening.

### Supplementary Note 15

The fingerprint imaging device according to any one of Supplementary Notes 1 to 3, further comprising:
one or more first imaging devices,
wherein each of the plurality of first imaging devices is disposed at a different position on an upper surface of the rotary table, and is moved to a position immediately below the opening as the rotary table is rotated.

### Supplementary Note 16

A fingerprint imaging device comprising:
a housing that has an upper surface in which an opening is formed:
a strut that is attached to the housing to be perpendicular to the upper surface of the housing:
a rotary table that is rotatably attached to the strut with the strut as a rotation axis: and
a plurality of first imaging devices disposed at different positions on the upper surface of the rotary table: wherein
a part of the rotary table is exposed from the housing, and
each of the plurality of first imaging devices is moved to a position immediately below the opening as the rotary table is rotated.

### Supplementary Note 17

The fingerprint imaging device according to Supplementary Note 16, wherein
at least one of the plurality of first imaging devices is an imaging device for imaging a fingerprint of a newborn baby,
the other first imaging device is imaging devices or the other first imaging devices are imaging devices for imaging a fingerprint of a target person other than a newborn baby, and
a resolution of the first imaging device for imaging the fingerprint of the newborn baby is higher than resolutions of the other first imaging device or the other first imaging devices.

### REFERENCE SIGNS LIST

1 FINGERPRINT IMAGING DEVICE
10 HOUSING
10a UPPER PORTION
10b LOWER PORTION
10c END PORTION
11 OPENING
12 STRUT
13 ROTARY TABLE
14 FIRST IMAGING DEVICE
14a FIRST IMAGING DEVICE
14b FIRST IMAGING DEVICE
15 SECOND IMAGING DEVICE
16 PROTECTIVE PLATE
17 CUTOUT PORTION
18 STRUT
20 GUIDE
21 CUTOUT PORTION
22 PROTRUSION
30 BEVEL GEAR
40 BEVEL GEAR
41 GEAR
50 DETECTION DEVICE
70 MOTOR CONTROL APPARATUS
71 PROCESSOR
72 COMMUNICATION INTERFACE
73 STORAGE DEVICE
80 ROTATION INSTRUCTION MEANS
81 PROTRUSION
82 GEAR
83 GEAR
84 STRUT
85 DISTAL END PORTION
90 ROTATION INSTRUCTION MEANS
91 PROTRUSION
92 GEAR
93 STRUT
94 COMPOSITE GEAR
95 ROTATION AXIS
96 COMPOSITE GEAR
97 STRUT
98 SPUR GEAR
100 MOTOR CONTROL PROGRAM
101 SIZE DETERMINATION UNIT
102 MOTOR CONTROL UNIT
200 NOTIFICATION CONTROL APPARATUS
201 PROCESSOR
210 NOTIFICATION CONTROL PROGRAM
211 IMAGING DEVICE DETERMINATION UNIT
212 NOTIFICATION CONTROL UNIT
220 COMMUNICATION INTERFACE
230 STORAGE DEVICE
300 NOTIFICATION CONTROL APPARATUS
301 PROCESSOR
310 NOTIFICATION CONTROL PROGRAM
311 SIZE DETERMINATION UNIT
312 IMAGING DEVICE SPECIFICATION UNIT
313 NOTIFICATION CONTROL UNIT
320 COMMUNICATION INTERFACE
330 STORAGE DEVICE
400 LIGHT SOURCE CONTROL APPARATUS
401 PROCESSOR
410 LIGHT SOURCE CONTROL PROGRAM
411 IMAGING DEVICE DETERMINATION UNIT
412 LIGHT SOURCE CONTROL UNIT
420 COMMUNICATION INTERFACE
430 STORAGE DEVICE

What is claimed is:

1. A fingerprint imaging device comprising:
a housing that has an upper portion in which an opening is formed;
a strut that is attached to the housing to be perpendicular to the upper portion of the housing;
a rotary table that is rotatably attached to the strut with the strut as a rotation axis;
a first imaging device that is disposed on an upper surface of the rotary table; and
a second imaging device that is disposed in the housing to be immediately below the opening and below the rotary table, wherein
a part of the rotary table is exposed from the housing, and
the first imaging device is moved to a position immediately below the opening as the rotary table is rotated.

2. The fingerprint imaging device according to claim 1, wherein
the first imaging device is an imaging device for imaging a fingerprint of a newborn baby,
the second imaging device is an imaging device for imaging a fingerprint of a target person other than a newborn baby, and
a resolution of the first imaging device is higher than a resolution of the second imaging device.

3. The fingerprint imaging device according to claim 1, wherein
the first imaging device and the second imaging device are imaging devices for imaging a fingerprint of a target person other than a newborn baby, and
a resolution of the first imaging device and a resolution of the second imaging device are the same.

4. The fingerprint imaging device according to claim 1, comprising:
a guide disposed on an upper surface of the housing to slide in a longitudinal direction of the fingerprint imaging device, wherein
the guide has a cutout portion formed over an upper surface and a lower surface of the guide, and
the cutout portion is formed such that a size of the cutout portion when viewed from an upper surface side of the guide is equal to or greater than a size of the opening.

5. The fingerprint imaging device according to claim 4, wherein
the rotary table is configured to be rotated in conjunction with sliding of the guide,
in a case where the guide is moved to a position where the cutout portion of the guide and the opening are continuous, the guide rotate the rotary table such that the first imaging device is moved to a position immediately below the opening, and
in a case where the guide at the position where the cutout portion of the guide and the opening are continuous is moved away from the opening, the guide rotate the rotary table such that fingerprint imaging of an imaging target by the second imaging device becomes possible.

6. The fingerprint imaging device according to claim 1, comprising:
a detection device that detects a size of a finger as an imaging target placed on the opening;
a motor that rotates the rotary table; and
a motor control apparatus that controls rotation of the motor, wherein
the motor control apparatus includes:
a memory configured to store instructions; and
a processor configured to execute the instructions to:
determine whether or not the size of the finger as the imaging target is equal to or smaller than a predetermined size, by using information detected by the detection device; and
transmit a first motor control signal to the motor in a case where the size of the finger as the imaging target is equal to or smaller than the predetermined size;
when the motor receives the first motor control signal, the motor rotates the rotary table such that the first imaging device is moved to a position immediately below the opening.

7. The fingerprint imaging device according to claim 6, wherein
the processor is configured to transmit a second motor control signal to the motor in a case where the size of the finger as the imaging target exceeds the predetermined size, and
when the motor receives the second motor control signal, the motor rotates the rotary table such that fingerprint imaging of the imaging target by the second imaging device becomes possible.

8. The fingerprint imaging device according to claim 1, further comprising:
extending member for rotating the rotary table, wherein
the extending member is configured to project from one side surface of the housing in a transverse direction of the fingerprint imaging device and to be movable in the transverse direction,
the extending member and the rotary table are connected via one or more gears, and
the extending member and the gears
rotate the rotary table such that the first imaging device is positioned immediately below the opening in a case where the extending member is pressed inward in the transverse direction of the fingerprint imaging device so that an exposed area of the extending member is minimized, and
rotate the rotary table such that fingerprint imaging by the second imaging device becomes possible in a case where the extending member is pulled outward in the transverse direction of the fingerprint imaging device from a state where the exposed area of the extending member is minimized and the exposed area of the extending member is not minimized.

9. The fingerprint imaging device according to claim 1, further comprising:
extending member for rotating the rotary table, wherein
the extending member is disposed to project from two side surfaces of the housing in a transverse direction of the fingerprint imaging device, and is movable in the transverse direction,
the extending member and the rotary table are connected via one or more gears, and
the extending member and the gears
rotate the rotary table such that the first imaging device is moved to a position immediately below the opening in a case where the extending member is pressed from a side of a first side surface of the two side surfaces, and
rotate the rotary table such that fingerprint imaging by the second imaging device becomes possible in a case where the extending member is pressed from a side of a second side surface different from the first side surface.

10. The fingerprint imaging device according to claim 1, wherein
the upper portion of the housing is attached to the housing to be rotatable about an end portion of the upper portion of the housing, on a side where the opening is not formed,
the upper portion of the housing includes extending member for rotating the rotary table,
the extending member is attached to the upper portion to be parallel to the strut,
the extending member and the rotary table are connected via one or more gears, and
the extending member and the gears
rotate the rotary table such that the first imaging device is moved to a position immediately below the opening in a case where the upper portion is closed, and
rotate the rotary table such that fingerprint imaging by the second imaging device becomes possible in a case where the upper portion is opened.

11. The fingerprint imaging device according to claim 1, comprising:
a notification apparatus that provides notification of which one of fingerprint imaging by the first imaging device and fingerprint imaging by the second imaging device is possible; and
a notification control apparatus that controls the notification apparatus, wherein
the notification control apparatus includes:
a memory configured to store instructions; and
a processor configured to execute the instructions to:
determine whether or not the first imaging device is positioned immediately below the opening on the basis of luminance of a captured image generated by the first imaging device or a rotation angle of the rotary table;
cause the notification apparatus to provide notification that fingerprint imaging by the first imaging device is possible in a case where it is determined that the first imaging device is positioned immediately below the opening; and
cause the notification apparatus to provide notification that fingerprint imaging by the second imaging device is possible in a case where it is determined that the first imaging device is not positioned immediately below the opening.

12. The fingerprint imaging device according to claim 1, comprising:
a notification apparatus that provides notification of whether or not correspondence between a finger as an imaging target placed on the opening and the first imaging device and the second imaging device is correct; and
a notification control apparatus that controls the notification apparatus, wherein
the notification control apparatus includes:
a memory configured to store instructions; and
a processor configured to execute the instructions to:
determine whether or not a size of the finger as the imaging target is equal to or smaller than a predetermined size, by using a captured image generated by the first imaging device or the second imaging device;
specify the imaging device that has imaged the finger as the imaging target, on the basis of the captured image used for determination of the size of the finger;
cause the notification apparatus to provide notification that the correspondence between the finger as the imaging target and the imaging device is correct in a case where the size of the finger as the imaging target is equal to or smaller than the predetermined size and the imaging device that has imaged the finger as the imaging target is the first imaging device or in a case where the size of the finger as the imaging target exceeds the predetermined size and the imaging device that has imaged the finger as the imaging target is the second imaging device; and
cause the notification apparatus to provide notification that the correspondence between the finger as the imaging target and the imaging device is incorrect in a case where the size of the finger as the imaging target is equal to or smaller than the predetermined size and the imaging device that has imaged the finger as the imaging target is the second imaging device or in a case where the size of the finger as the imaging target exceeds the predetermined size and the imaging device that has imaged the finger as the imaging target is the first imaging device.

13. The fingerprint imaging device according to claim 1, comprising:
a first light source and a second light source that irradiate a finger as an imaging target placed on the opening; and
a light source control apparatus that controls the first light source and the second light source, wherein
a light amount of the second light source is greater than a light amount of the first light source,
the light source control apparatus includes:
a memory configured to store instructions; and
a processor configured to execute the instructions to:
determine whether or not the first imaging device is positioned immediately below the opening on the basis of luminance of a captured image generated by the first imaging device or a rotation angle of the rotary table;
turn on the first light source in a case where it is determined that the first imaging device is positioned immediately below the opening; and
turn on the second light source in a case where it is determined that the first imaging device is not positioned immediately below the opening.

14. The fingerprint imaging device according to claim 1, comprising:
one light source that irradiates a finger as an imaging target placed on the opening; and
a light source control apparatus that controls the light source, wherein
the light source control apparatus includes:
a memory configured to store instructions; and
a processor configured to execute the instructions to:
determine whether or not the first imaging device is positioned immediately below the opening on the basis of luminance of a captured image generated by the first imaging device or a rotation angle of the rotary table;
turn on the light source with a predetermined light amount in a case where it is determined that the first imaging device is positioned immediately below the opening; and
turn on the light source with a light amount greater than the predetermined light amount in a case where it is determined that the first imaging device is not positioned immediately below the opening.

15. The fingerprint imaging device according to claim 1, further comprising:
one or more first imaging devices,
wherein each of the plurality of first imaging devices is disposed at a different position on an upper surface of the rotary table, and is moved to a position immediately below the opening as the rotary table is rotated.

16. A fingerprint imaging device comprising:
a housing that has an upper surface in which an opening is formed;
a strut that is attached to the housing to be perpendicular to the upper surface of the housing;
a rotary table that is rotatably attached to the strut with the strut as a rotation axis; and
a plurality of first imaging devices disposed at different positions on the upper surface of the rotary table; wherein
a part of the rotary table is exposed from the housing, and
each of the plurality of first imaging devices is moved to a position immediately below the opening as the rotary table is rotated.

17. The fingerprint imaging device according to claim 16, wherein
at least one of the plurality of first imaging devices is an imaging device for imaging a fingerprint of a newborn baby,
the other first imaging device is imaging devices or the other first imaging devices are imaging devices for imaging a fingerprint of a target person other than a newborn baby, and
a resolution of the first imaging device for imaging the fingerprint of the newborn baby is higher than resolutions of the other first imaging device or the other first imaging devices.
